(19) United States
(12) Patent Application Publication
(10) Pub. No.: US 2020/0371110 A1
(43) Pub. Date: Nov. 26, 2020

(54) METHODS FOR IDENTIFYING AND TREATING HIV PERSISTENCE

(71) Applicant: The Wistar Institute of Anatomy and Biology, Philadelphia, PA (US)

(72) Inventor: Mohamed ABDEL-MOHSEN, Wynnewood, PA (US)

(21) Appl. No.: 16/961,043

(22) PCT Filed: Jan. 11, 2019
(86) PCT No.: PCT/US19/13128

§ 371 Date: Jul. 9, 2020

Related U.S. Application Data
(60) Provisional application No. 62/616,695, filed on Jan. 12, 2018.

(51) Int. Cl. G01N 33/66; G01N 33/569; A61K 9/51; A61K 31/715; A61K 47/42

(52) U.S. Cl. CPC G01N 33/66; G01N 33/56983; A61K 47/42; A61K 31/715; A61K 9/5123

(57) ABSTRACT

Methods for identifying or monitoring or treating HIV persistence or the development of an HIV-comorbidity in an HIV+ subject involve certain selected glycan dysregulations. In certain embodiments, hyposialylation in the total IgG glycome or total plasma glycome of an HIV+ subject during or after antiretroviral therapy is an indication of HIV persistence and can be predictive of developing co-morbidities. Methods of treating HIV persistence or preventing developing co-morbidities involves modifying or manipulating the selected glycan by administering therapeutic agents that will modify the levels of the selected glycan, a precursor thereof, or another component of its pathway.

### CROSS-REFERENCE TO RELATED APPLICATIONS

This application claims the benefit of the priority of U.S. Provisional Patent Application No. 62/616,695, filed Jan. 12, 2018, which application is incorporated herein by reference.

### STATEMENT REGARDING FEDERALLY SPONSORED RESEARCH OR DEVELOPMENT

This invention was made with government support under grant nos. R21 AI129636, R21 NS106970, P30 AI 045008, U01 A1065279 and UM1 AI126620, awarded by the National Institutes of Health. The government has certain rights in this invention.

### BACKGROUND OF THE INVENTION

Antiretroviral therapy (ART) has dramatically reduced morbidity and mortality in HIV+ individuals. However, ART requires lifelong administration and does not eradicate HIV. Persistent HIV infection continues to cause immune activation, chronic inflammation, and damage to multiple organs. Despite suppression of viral replication with ART, HIV infection remains associated with significantly increased morbidity and mortality in those with elevated immune activation and poor CD4+ T cell recovery. Specifically, CD4+ T cell counts above 350 cells/μl portend a near normal lifespan; whereas suboptimal CD4+ T cell recovery presages a considerably shorter life expectancy.¹ In addition, HIV infection is associated with chronic inflammation, which in turn is associated with higher risk of mortality in HIV infected individuals.²

Patients who achieve virologic suppression on ART, but have incomplete reconstitution of CD4+ T cell counts, are termed immunological non-responders (INRs). INRs, who make up about 20% of HIV+individuals, have an about 10-fold increased risk of an AIDS-defining event, i.e., inflammation-associated comorbidity or death, compared to immunological responders (IRs)^{3,4}. The latest US Health and Human Services HIV/AIDS treatment guidelines have noted the importance to study the INR phenotype. However, due to lack of understanding of the causes of this phenotype, no effective treatment or monitoring is available or recommended for those at higher risk of death due to poor CD4+ T cell recovery.²²

It is increasingly appreciated that immune responses are modulated by the host glycome. Host glycans interact with their binding proteins to influence cell-cell interactions.⁵⁻⁷ Certain disease states have been associated with aberrant glycosylation patterns, e.g., with the loss of the monosaccharide sialic acid (hypo-sialylation) from cell surfaces and from plasma circulating glycoproteins including immunoglobulin G (IgG)⁸⁻¹⁶

Global antibody glycosylation is dynamic and plays critical roles in shaping different immunological outcomes and direct antibody functionality during HIV infection. Levels of anti-HIV-1 antibodies can reflect the degree of HIV persistence and low-level viral replication. HIV infection has been associated with certain changes in the antibody glycosylation, mainly higher levels of agalactosylated antibodies^{58,59}. Changes in global and antigen-specific antibody glycosylation have been associated with a differential activity of anti-HIV antibodies to control HIV infection⁵⁸. However, the relevance of glycosylation patterns to HIV persistence after antiretroviral therapy (ART) or to HIV reservoir size or the development of other co-morbidities after ART in vivo remains unknown.

### SUMMARY OF THE INVENTION

In one aspect, an in vitro method for identifying or monitoring or treating HIV persistence or the development of an HIV-comorbidity in an HIV+ subject is provided. The methods include generating a glycomic signature characterized by the level of selected single glycan structure or multiple glycan structures within a biological sample obtained from the HIV+ subject and or within a component of the sample. The signature is then analyzed for identification of the levels of certain glycan structures within the sample compared with that from a control. Selected modification of the glycomic signature provides an indication of developing an HIV-comorbidity. In certain embodiments of this method, the subject has received antiretroviral therapy (ART) before or during the occurrence of the disease.

In another aspect, methods for treating an HIV-infected subject comprise modifying or normalizing the level of a selected glycan structure in the subject's glycome. In one aspect, the method involves increasing the level of a selected glycan to that of an uninfected control or an Immune Responder control or a control negative for an HIV comorbidity. In another aspect, the method involves decreasing the level of a selected glycan to that of an uninfected control or an Immune Responder control or a control negative for an HIV comorbidity.

Other aspects and advantages of these compositions and methods are described further in the following detailed description of the preferred embodiments thereof

### BRIEF DESCRIPTION OF THE DRAWINGS

FIGS. 1A-1E provide evidence of HIV-associated glycomic alterations in total IgG glycome. FIG. 1A is a heatmap describing alterations in total IgG glycome associated with unsuppressed and suppressed HIV infection. Changes of levels are represented by grayscale while higher levels are marked with “+” and lower levels are blank. S2=percentage of di-sialylated structures, F=percentage of all fucosylated structures, G0=percentage of agalactosylated structures, G2=percentage of di-galactosylated structures, S1=percentage of monosialylated structures, G1=percentage of monogalactosylated structures, and B=percentage of structures with bisecting GlcNAc. FIG. 1B shows the percentage of di-sialylated glycan structures in total IgG glycome. FIG. 1C shows the percentage of A2G2S2 glycan trait in total IgG glycome. FIG. 1D shows the percentage of fucosylated glycan structures in total IgG glycome. FIG. 1E shows the percentage of agalactosylated glycan structures in total IgG glycome. All statistical comparisons were performed using two-tailed non-parametric Mann-Whitney t test. Error bars represent median and interquartile range (IQR).

FIGS. 2A-2G show the HIV-associated glycomic alterations in total plasma glycome. FIG. 2A shows a heatmap describing alterations in total plasma glycome associated with unsuppressed and suppressed HIV infection. Changes of levels are represented by grayscale while higher levels are marked with “+” and lower levels are blank. S0=neutral glycan structures, S1=monosialylated structures, S2=di-sialylated structures, S3=trisialylated structures, S4=tetrasialylated structures, G0=agalactosylated structures, G1=monogalactosylated structures, G2=di-galactosylated structures, G3=trigalactosylated structures, G4=tetragalactosylated structures, LB=low branched (monoantennary and diantennary) structures, HB=high branched (triantennary and tetraantennary), B=structures with bisecting GlcNAc, FUC-A=antennary fucosylated structures, and FUC-C=core fucosylated structures. FIG. 2B shows percentage of structures with bisecting GlcNAc in total plasma glycome. FIG. 2C shows percentage of agalactosylated glycan structures in total plasma glycome. FIG. 2D shows percentage of core-fucosylated glycan structures in total plasma glycome. FIG. 2E shows the percentage of neutral glycan structures in total plasma glycome. FIG. 2F shows the percentage of di-sialylated glycan structures in total plasma glycome. FIG. 2G shows the percentage of di-galactosylated glycan structures in total plasma glycome. All statistical comparisons were performed using two-tailed non-parametric Mann-Whitney t test. Error bars represent median and interquartile range (IQR).

FIGS. 3A-3C show that the levels of certain glycomic traits, e.g., IgG galactosylation, in isolated IgG glycomes correlate with HIV persistence during ART. FIG. 3A shows in four graphs correlations between percentage of A2G1 in total IgG glycomes of HIV+ ART-suppressed individuals and HIV DNA of unfractionated PBMC, HIV RNA in unfractionated PBMC, HIV DNA in isolated CD4+ T cells, and HIV RNA in isolated CD4+ T cells. FIG. 3B shows correlations between percentage of A2G2 in total IgG glycomes of HIV+ ART-suppressed individuals and the same four HIV DNA or HIV RNA measurements as in FIGS. 3A. FIG. 3C shows percentage of A2BG2, in total IgG glycomes of HIV+ ART-suppressed individuals and the same four HIV DNA or HIV RNA measurements. P-values were obtained using two-tailed non-parametric Spearman's rank tests.

FIGS. 4A-4C show that the levels of certain glycomic traits in total plasma glycomes correlate with HIV persistence during ART. FIG. 4A shows the correlations between unfractionated PBMC and CD4+ T cell-associated HIV DNA and RNA and percentage of A2[6]BG1 in total plasma glycomes of HIV+ ART-suppressed individuals. FIG. 4B shows the same percentage correlations for A2G2. FIG. 4C shows the same percentage correlations for A2BG2. P-values were obtained using two-tailed non-parametric Spearman's rank tests.

FIG. 5 shows that the percentage of A2G2 glycan trait in total IgG glycome is higher in ART-suppressed individuals. Percentage of A2G2 glycan trait in total IgG glycome. Statistical comparisons were performed using non-parametric two-tailed Mann-Whitney t test. Error bars represent median and interquartile range (IQR). FIGS. 6A-6H show that the levels of certain circulating anti-inflammatory glycans associate with higher levels of CD4 count and lower levels of T cell activation. FIG. 6A shows correlations between levels of A4G4S4 glycan trait in total plasma glycomes of HIV+ ART-suppressed individuals and CD4 count. FIG. 6B shows correlations between levels of A4G4S4 glycan trait in total plasma glycomes of HIV+ ART-suppressed individuals and CD4%. FIG. 6C shows correlations between levels of A4G4S4 glycan trait in total plasma glycomes of HIV+ ART-suppressed individuals and percentage of CD4+ T cells expressing the HLA-DR late activation marker. FIG. 6D shows correlations between levels of A4G4S4 glycan trait in total plasma glycomes of HIV+ ART-suppressed individuals and percentage of CD4+ T cells expressing the intermediate or late CD25 activation marker. FIG. 6E shows levels of FA3G3S3 glycan trait in total plasma glycomes of HIV+ ART-suppressed individuals and CD4%. FIG. 6F shows levels of FA3G3S3 glycan trait in total plasma glycomes of HIV+ ART-suppressed individuals and percentage of CD4+ T cells expressing the intermediate or late CD25 activation marker. FIG. 6G shows the levels of FA2BG2S1 glycan trait in total IgG glycomes of HIV+ ART-suppressed individuals and CD4 count. FIG. 6H shows the levels of A2BG2 glycan trait in total IgG glycome of HIV+ ART-suppressed individuals and percentage of CD4+ T cells expressing the CD69 early activation marker. P-values were obtained using two-tailed non-parametric Spearman's rank tests.

FIG. 7A shows that IgG N-glycan samples were all separated into 24 peaks.

FIG. 7B shows that total plasma N-glycans were all separated into 39 peaks.

FIGS. 8A-8F show a lack of correlation between the levels of glycan traits correlated with HIV persistence during ART and age. FIGS. 8A-8C show correlations between levels of A2G1, A2G2, and A2BG2 in total IgG glycome and age, respectively. FIGS. 8D-8F show correlations between levels of A2[6]BG1, A2G2, and A2BG2 in total plasma glycome and age, respectively. P-values were obtained using two-tailed non-parametric Spearman's rank tests.

FIGS. 9A-9D show that hypo-sialyation persists in HIV+individuals despite long-term suppressive ART. HIV+ plasma binds less to lectins (FIG. 9A shows lectin SSA; and FIG. 9B shows lectin SNA) that are specific to sialylated glycans, despite suppressive ART. FIG. 9C shows results with a tri/tetra antennary complex type N-glycan (or hypo-sialylated N-linked glycoproteins; PHA-L). FIG. 9D shows results with an α- or β-linked terminal GalNAc (or hypo-sialylated O-linked glycoproteins) SBA.

FIGS. 10A-10B show that HIV-infection is associated with persistent loss of sialylated glycans in total IgG glycome. FIG. 10A shows the percentage of di-sialylated glycan structures in total IgG glycome. FIG. 10B shows the percentage of A2G2S2 glycan trait in total IgG glycome. All comparisons were performed using two-tailed non-parametric Mann-Whitney t-test. Error bars represent median and interquartile range.

FIGS. 11A-11D show that levels of certain circulating anti-inflammatory glycans associate with higher levels of CD4 count and lower levels of T cell activation. FIGS. 11A through 11D show correlations between levels of A4G4S3 glycan trait in total plasma glycome and CD4 count, CD4%, percentage of CD4+ T cells expressing the HLA-DR late activation marker, and percentage of CD4+ T cells expressing the intermediate or late CD25 activation marker, respectively.

FIGS. 12A-12B show that levels of glycomic traits in isolated IgG glycomes correlate with HIV persistence during ART. FIGS. 11A and 11B show correlations between unfractionated PBMC and either CD4+ T cell-associated HIV DNA and RNA, respectively, vs. percentage of A2BG2, in total IgG glycomes. P-values were obtained using two-tailed non-parametric Spearman's rank tests.

FIG. 13 is a graph showing that certain cell-free glycan structures associate with cardiovascular disease risk during HIV infection.

FIG. 14 is a schematic overview of the study testing that HIV-associated hypo-sialylation is linked to poor immune reconstitution and chronic inflammation after ART in vivo.

FIG. 15 shows that primary human monocytes were stimulated with LPS (10 ng/ml)±nude nanoparticles or sialic acid coated nanoparticles (50 μg/ml) for 18 hours. Supernatants were assayed for TNF-α by ELISA. Statistical significance was assessed by Mann-Whitney U test.

FIG. 16 shows a chemical reaction for obtaining sialic-acid coated (functionalized) nanoparticles. Generally, about 50 μg sialic acid/mg of PLGA nanoparticles was used in the experiments of FIGS. 17A-17E.

FIGS. 17A-17E show that in an experiment, sialic acid coated nanoparticles prevent immune activation and exhaustion in HIV-infected humanized BLT mice.

FIG. 17A is a graph plotting % CD3+CD8+HLA-DR+CD38+ vs. time. The symbol H represented the 3 times of administrations of 100 μg/sialic acid/mouse as the injection concentration.

FIG. 17B plots % CD3+CD8+HLA-DR+CD38+ vs CD8 T cell activation. In each column representing a different tissue of origin, the leftmost horizontal square represents nude nanoparticles and the rightmost horizontal square (or line) represents the sialic-acid coated nanoparticles.

FIG. 17C plots % CD3+CD8+PD1+ vs PD1 expression on CD8 T cells. In each column representing a different tissue of origin, the leftmost horizontal square (or line) represents nude nanoparticles and the rightmost horizontal square (or line) represents the sialic-acid coated nanoparticles.

FIG. 17D shows an HLA-DR mean fluorescence intensity (MFI) of the nanoparticles on monocytes. Leftmost curve is sialic acid coated nanoparticles; rightmost curve is nude nanoparticles.

FIG. 17E shows the MFI of HLA-DR in human monocytes from lung). Bars are as labeled.

### DETAILED DESCRIPTION OF THE INVENTION

The methods and compositions described herein relate to the use of certain glycomic alterations as biomarkers of HIV persistence and the development of HIV co-morbidities after antiretroviral (ART) therapy. Also provided are novel HIV-treatment strategies based on reversing or modifying the adverse impact of HIV+ subjects' glycomic dysregulation on immune reconstitution. As disclosed herein, novel plasma and IgG glycomic alterations that are associated with suppressed HIV infection as well as with CD4 T cell lymphocyte levels of HIV DNA and RNA during suppressive antiretroviral therapy are provided. Additionally, as provided herein, the inventors have observed that the ability of antibody glycosylation to impact several immunological responses, including ADCC and immune activation/inflammation, plays a role in defining HIV reservoir size during ART as well as in novel treatments directed at reducing HIV reservoir size during ART.

Our data support that HIV-induced hypo-sialylation persists despite long-term suppressive art and correlates with immune activation, CD4+ T cell count, and the development of HIV-associated comorbidities. Here we demonstrate that glycomic dysregulation, as exemplified by hypo-sialylation, contributes to the INR phenotype.

As discussed below, data show that HIV induces a hypo-sialylation state that persists despite long-term suppressive ART, and provide evidence that this glycomic dysregulation is linked to poor immune reconstitution and chronic inflammation, CD4+ T cell count, and the development of other HIV-associated comorbidities after ART in vivo. The methods and compositions disclosed herein rely on the ability to detect and ameliorate glycomic dysregulation, e.g., hypo-sialylation, and its mediation of immune activation, inflammation, and immune reconstitution after ART.

### Definitions and Components Used in the Methods

Unless defined otherwise in this specification, technical and scientific terms used herein have the same meaning as commonly understood by one of ordinary skill in the fields of biology, biotechnology and molecular biology and by reference to published texts, which provide one skilled in the art with a general guide to many of the terms used in the present application. The definitions herein are provided for clarity only and are not intended to limit the claimed invention.

The term “anti-retroviral therapy” or “ART” refers to treatment of individuals infected with human immunodeficiency virus (HIV) using anti-HIV drugs. The standard treatment consists of a combination of at least three drugs (often called “highly active antiretroviral therapy” or HAART) that suppress HIV replication. Antiretroviral medicines that are often used to treat HIV include: Nucleoside/nucleotide reverse transcriptase inhibitors, also called nucleoside analogs, such as abacavir, emtricitabine, and tenofovir. These medicines are often combined for best results. Nonnucleoside reverse transcriptase inhibitors (NNRTIs), such as efavirenz, etravirine, and nevirapine. Protease inhibitors (PIs), such as atazanavir, darunavir, and ritonavir. Entry inhibitors, such as enfuvirtide and maraviroc. Integrase inhibitors, such as dolutegravir and raltegravir. In one embodiment, ART is a combination of drugs efavirenz, tenofovir, and emtricitabine. Other combinations, without limitation, include: Dolutegravir, abacavir and lamivudine, Dolutegravir, tenofovir and emtricitabine, elvitegravir, cobicistat and tenofovir, and emtricitabine, raltegravir, tenofovir and emtricitabine, or ritonavir-boosted darunavir, tenofovir and emtricitabine.

The term “glycan” as used herein refers to a complex oligosaccharide composed of 10-15 monosaccharide residues. One or more glycan(s) can be covalently attached to a protein to form a glycoprotein(s), or to a lipid(s) to form a glycolipid(s). Most human proteins are modified by covalent attachment of glycans. Most glycans attached to proteins can be classified as N-glycans, attached through nitrogen of asparagine, or O-glycans, attached through oxygen of mainly serine or threonine. Glycans of interest for use in the glycomic signatures can include, without limitation, one or more of monosialylated structures, di-sialylated structures, trisialylated structures, tetrasialylated structures, agalactosylated structures, monogalactosylated structures, di-galactosylated structures, trigalactosylated structures, tetragalactosylated structures, low branched (monoantennary and diantennary) structures, high branched (triantennary and tetraantennary), structures with bisecting GlcNAc, antennary fucosylated structures, and core fucosylated structures.

The term “glycome” as used herein refers to the set of all glycans in an organism/tissue/cell, or even of a single glycoprotein. In one embodiment, the glycome is the set of all glycans in the IgG of a human subject. In another embodiment, the glycome is the set of all glycans in the plasma of a human subject. In another embodiment, the glycorne is the set of all glycans on the subject's cell-surface, either from all cells or from a selected cell type. In another embodiment, the glycome is the set of all glycans in the subject's tissues, either from all tissues or from a selected tissue type. In another embodiment, the glycome is the subject's total exosome-bound glycome.

The term “lectin” refers to a protein with a functional carbohydrate recognition domain which binds specific glycan structures, regarding both monomer composition and spatial arrangement.

The term “glycomics” refers to the collection, analysis, and exploitation of glycol biological data at the glycome level. Glycomics studies in a cell or organism level can provide a general overview on the glycome, the total glycosylation pattern of glycoproteins, lipids, or other types of biomolecules. In one aspect, the total glycome is obtained from plasma. In another aspect the total IgG glycome is analyzed.

The term “glycomic profiling” permits the identification of a set of, or all, N-glycans expressed by plasma/serum, cell tissue or organism. In one protocol, all glycans attached to the proteins are released by enzymatic digestion, and then separated by hydrophilic chromatography and finally quantitatively profiled with MALDI-TOF MS system (Creative Proteomics, Shirley, NY). Other methods are known for glycomic profiling. Methods for glycomic profiling are described in detail in US published patent application Nos. 2012/0276560, 2009/0029343, International patent publication No. WO2012/082830, U.S. Pat. No. 9,772,337; and in the extensive literature cited in the references. The methods and techniques provided in these references are incorporated herein by reference to supplement the teachings of this specification.

The term “glycomic signature” or “glycomic profile” as used herein refers to a pattern of one or more, or total glycosylated proteins or antibodies present in a biological sample of a human. A glycomic signature can be characteristic of a healthy state or a disease or disease state. In one embodiment, a glycomic signature characterized by hypo-sialylation is a determined to be a characteristic of an INR subject, or an HIV+ subject having a likelihood of HIV persistence or developing one of the HIV-comorbidities as discussed herein. Still other glycomic signatures include modifications of one or more specified glycans as characteristic of the morbidity of cardiac diseases or neurological impairment.

The term “glycomic dysregulation” as used herein means a modification of a glycosylation or glycosylation pattern of the glycome of a biological sample that differs from that found in a control, which in one aspect is a normal healthy control and, in another aspect, is an IR post-ART. Still other controls, e.g., IR controls or controls negative for an HIV comorbidity, may be used to generate and define glycomic signatures.

The term “glycosylation” refers to the modification of a protein by addition of a sugar molecule. Glycosylation can alter an antibody's capacity to interact with classical and non-classical Fc receptors, which defines the antibody functionality in inducing antibody-dependent cell-mediated cytotoxicity (ADCC), antibody-dependent cellular phagocytosis (ADCP), complement dependent cytotoxicity (CDC), and anti-inflammatory activities²⁷⁻³⁰. Higher levels of antibody galactosylation (i.e. modification caused by addition of galactose sugar units) and lower levels of fucosylation (modification caused by addition of fucose sugar units) are associated with a higher ADCC function of antibodies. Antibody sialylation (i.e., modification by additional of a sialyl group) and galactosylation are linked to strong anti-inflammatory responses¹³⁻¹⁶; however, the exact mechanism underlies this effect is not clear. It has been suggested that it could be related to either switching the antibody's binding from classic Fc receptors to non-classical Fc receptors, or binding to sialic-acid-binding proteins (siglecs) on the surface of monocytes and macrophages, thus initiating an inhibitory signal that leads to an anti-inflammatory response through inhibition of TLR4 signal transduction³¹⁻³⁴.

“Patient” or “subject” or “individual” as used herein means a mammalian animal, including a human, a veterinary or farm animal, a domestic animal or pet, and animals normally used for clinical research. In one embodiment, the subject of these methods and compositions is a human. In the discussion of HIV treatment with anti-retroviral therapy, there are two classes of subjects: Immunological Non-Responders (INRs) are an approximately 20% of HIV+ subjects who are characterized by achieving virologic suppression on ART, but have incomplete reconstitution of CD4+ T cell counts. A number of factors associated with the INR phenotype have been identified: time >1 year from first CD4+ T cell count <200 cells/μl,⁴ advanced age,^{4,17} strong CMV-specific T cell responses¹⁷ and low nadir CD4+ T cell count.¹⁸ Some studies have found that activation of CD4+ and CD8+ T cells is associated with the INR phenotype^{19,20} though not all found a correlation with CD8+ T cell activation²¹. In one embodiment INRs are characterized by a CD4 cell count of <350 cells/μl for more than 2 years after administration of ART. Immunological Responders (IRs) are HIV+ subjects who achieve virologic suppression on ART with immune reconstitution. In one embodiment, IRs are characterized by a CD4 cell count of >500 cells/μl for more than 2 years after administration of ART.

“Sample” as used herein means any biological fluid or suspension or tissue from a subject, that contains glycomic biomarkers or a glycomic signature as identified herein. The most suitable samples for use in the methods and with the diagnostic compositions or reagents described herein are samples or suspensions which require minimal invasion for testing, e.g., total plasma or isolated immunoglobulin G (IgG). Other samples which can be manipulated for measurement of glycomic biomarkers include blood samples, including whole blood, peripheral blood, or serum, as well as cerebrospinal fluid, serous fluid, saliva or urine, vaginal or cervical secretions, and ascites fluids or peritoneal fluid or tissues containing HIV reservoirs. In another embodiment, a suitable sample for use in the methods described herein includes peripheral blood, more specifically peripheral blood mononuclear cells. Any sample containing the glycomic biomarkers may be similarly evaluated by the methods described herein. In one embodiment, such samples may further be treated to isolate the indicated glycoproteins modified by selected glycans. Alternatively, such samples are treated to isolate total glycans modifying all proteins. In another embodiment, the samples are concentrated by conventional means.

Control, control level, control signature or control profile as used herein refers to the source of the reference glycomic signature against which the tested subject's glycomic signature is analyzed, i.e., the levels of one or more selected glycans or total plasma, IgG, circulating, or HIV reservoir glycomes in a specified subject or in an average population of multiple subjects having a common condition or stage of disease. In one embodiment, the reference glycomic signatures are obtained from biological samples selected from a reference healthy non-HIV-infected human subject or average population of such subjects. In another embodiment, the reference glycomic signature utilized is a signature or profile derived from biological samples of a reference human subject or population of human subjects who are post-ART and demonstrate no HIV comorbidities and/or no HIV reservoirs. In one embodiment the reference population comprises Immune Responders (IR). In another embodiment the reference population comprises Immune Non-Responders (INR). In certain embodiments, the reference glycomic signature or profile utilized is a profile derived from a reference human subject, or an average of multiple subjects, with specific early stage co-morbidities. In another embodiment, the reference signature or profile is a standard or profile derived from a reference human subject, or an average of multiple subjects, with late stage co-morbidities. In another embodiment, the reference glycomic signature is a profile derived from the biological samples of the same human subject at a prior time, e.g., before or after ART, or before or after treatment with a therapeutic agent for manipulating the subject's glycomic signature. The control or reference standard, in various embodiments, is a mean, an average, a numerical mean or range of numerical means, a numerical pattern, a graphical pattern or a nucleic acid or gene expression profile derived from a control subject or a control population.

As used herein, the term “treatment” refers to any method used to alleviate, delay onset, reduce severity or incidence, or yield prophylaxis of one or more symptoms or aspects of a disease, disorder, or condition. For the purposes of the present invention, treatment can be administered before, during, and/or after the onset of symptom. In certain embodiments, treatment occurs after the HIV+subject has received ART. In some embodiments, the term “treating” includes abrogating, substantially inhibiting, slowing, or reversing the progression of a condition, substantially ameliorating clinical or aesthetical symptoms of a condition, or substantially preventing the appearance of clinical or aesthetical symptoms of a condition, or decreasing the severity and/or frequency one or more symptoms resulting from the disease. More specifically treatment includes manipulating the level of the selected glycan to reduce HIV+ persistence, decrease the HIV+ reservoir, and/or reduce the severity, delay the onset, or prevent the development of a HIV comorbidity. In one embodiment, where the glycan is sialic acid, treatment involves administering sialic acid-containing compositions or therapeutic agents that operate to increase the levels of sialic acid in the subject. In another embodiment, where the glycan is fucose, treatment involves administering compositions or therapeutic agents that operate to decrease the levels of fucose in the subject.

By “therapeutic agent” as used herein means any compositions that can be used to manipulate the host glycome, including modifying the levels of one or multiple glycans in the subject's total glycome to reduce the HIV reservoir, ameliorate HIV persistence and treat or delay the onset of HIV co-morbidities. In one embodiment, the therapeutic agent is a selected glycan in or associated with a suitable pharmaceutical carrier or excipient, such as a nanoparticle coated with one or more glycans. As discussed herein, and in the examples below, a sialic acid coated nanoparticle may be administered to a subject demonstrating hyposialylation in the total IgG glycome or total plasma glycome indicative of HIV persistence or one or the co-morbidities. In another embodiment, the therapeutic reagent is a conjugate formed of a targeting moiety and the glycan. In still another embodiment, the therapeutic reagent can include a compound or precursor in the selected glycan biosynthetic pathway or derivative thereof, e.g., for example a precursor in the sialic acid pathway. In still another embodiment, the therapeutic reagent is an inhibitor of the glycan or precursor in the glycan biosynthetic pathway or derivative thereof or a glycosylation inhibitor or deglycosylation enzyme, which can reduce the over-production of the selected glycan. Still other therapeutic reagents can include compounds or chemical moieties that can manipulate glycosyltransferase expression. Any of the active therapeutic reagents can be associated with known carriers or targeting compositions, such as taught in the prior art.

By “HIV co-morbidity” is meant to include, without limitation, an age-associated disease, inflammation-associated disease, and immune-activation-associated disease which occurs in HIV+ human subjects at an earlier time or progressing more rapidly to an advanced stage, or occurring at a more aggressive and dangerous stage than in the general HIV-uninfected population. In one embodiment, the co-morbidity is a cardiovascular disease. In another embodiment, the disease is a neurological impairment. In still another embodiment, the co-morbidity is a cancer. In still another embodiment, the cancer is an AIDS-defining cancer, such as Kaposi's sarcoma. In another embodiment, the comorbidity is a metabolic disorder. In still another embodiment, the co-morbidity is a kidney disease, a liver disease, a lung disease, or a bone disease.

Throughout this specification, the words “comprise”, “comprises”, and “comprising” are to be interpreted inclusively rather than exclusively. The words “consist”, “consisting”, and its variants, are to be interpreted exclusively, rather than inclusively. It should be understood that while various embodiments in the specification are presented using “comprising” language, under various circumstances, a related embodiment is also described using “consisting of” or “consisting essentially of” language.

The term “a” or “an”, refers to one or more, for example, “a biomarker,” is understood to represent one or more biomarkers. As such, the terms “a” (or “an”), “one or more,” and “at least one” are used interchangeably herein.

As used herein, the term “about” means a variability of 10% from the reference given, unless otherwise specified.

### Methods

In one aspect, a method is described herein based upon the novel observations and correlations made by the inventors for identifying or monitoring or treating HIV persistence in a subject. The subject in one embodiment is an HIV+ subject who has received or currently is receiving antiretroviral therapy (ART). The method can evaluate the subject before or during the occurrence of active AIDS or before or during the occurrence of an HIV comorbidity. In another embodiment, the subject is an immunological non-responder (INR). Still other subjects may be evaluated and treated by these methods.

The methods involve identifying, monitoring treating, retarding, or preventing the development of one or more HIV-comorbidities in an HIV+ subject. In one embodiment, the methods involve first obtaining a biological sample from the HIV+ subject. The biological sample, e.g., whole blood, contains glycosylated proteins and lipids. A component of whole blood, e.g., plasma or PBMCs, may be further purified from other components in the sample that are unnecessary for the analysis. The sample or components of the sample is manipulated to generate a glycomic signature characterized by the level of selected single glycan structure or multiple glycan structures. As one example, the sample may be manipulated to provide the subject's total plasma glycome from which the selected glycan(s) can be measured. In another embodiment, the sample is manipulated to provide the subject's total IgG glycome. Still other cell-containing samples can be manipulated to provide the subject's cell-surface glycome. The cell-surface glycome can be generated from a selected cell type in a subject. Alternatively, a cell surface glycome can be measured from a collection of multiple cell types. A global cell surface glycome can also be measured from a collection of all cell types in the subject. Still another embodiment generates the subject's total exosome-bound glycome.

Treatment of the selected sample to provide measurable and identifiable glycans can use techniques as described.⁶¹ See also, US patent publication No. 2016/0103137, published Apr. 13, 2016, incorporated by reference herein, for additional method steps for preparing and measuring glycans from a plasma or IgG sample. According to the methods the glycoprotein(s) in the sample are isolated and purified. Isolation and purification methods are known in the art, for example, SDS PAGE, size exclusion chromatography, affinity resin or beads, filtration/isolation columns, various centrifugation methods to separate fractions, and the like.

In one embodiment, glycans can be separated from the pooled glycoproteins from the sample or sample component for measurement by use of treatment or digestion with an appropriate glycosidase. Individual glycans can be further segregated by use of ligands (labeled or immobilized on a solid support), such as antibodies, lectins; and then subjected to affinity purification and high-throughput analysis by HPLC. The glycan level in the test sample is determined by one or more of the following techniques, i.e., which include high-performance liquid chromatography (HPLC; e.g., normal phase or weak anion exchange HPLC), capillary electrophoresis (CE), gel electrophoresis (e.g., one or two dimensional gel electrophoresis), mass spectrometry (MS), isoelectric focusing (IEF), lectin-based microarray chromatography and/or an immunoassay (e.g., immuno-PCR, ELISA, lectin ELISA, Western blot, or lectin immunoassay) on the sample or a component thereof (e.g., plasma, IgG, serum, PBMCs or tissue lysate, a pool of isolated glycans, an isolated glycoprotein, etc.). See, e.g., U.S. patent application publications 20060269974; 20060270048 and 20060269979. In one embodiment, methods for generating a glycomic signature, and/or measuring a selected single glycan structure or multiple glycan structures within a sample can use ultra-performance liquid chromatography (UPLC), as described in the examples and references below. In other embodiments, electrospray ionization—time of flight (ESI-TOF) MS coupled with reversed-phase (RP) HPLC or size-exclusion chromatography (SEC) is used. Still other techniques include matrix assisted laser desorption ionization (MALDI) MS.⁶⁰

Once the level of the selected glycan or multiple glycans are determined which generated the glycomic signature, analysis by comparison to the selected control is performed. In one embodiment for analyzing the glycomic signature or profile of the tested subject, the level of the selected glycan(s) is determined with reference to a selected control, i.e., one of the controls identified above. Reduced levels of the selected glycan(s) or increased levels of the selected glycan(s) in comparison to the control can be any level having statistical significance from a level of the selected control. In certain embodiments, levels of a selected glycan, e.g., sialic acid or fucose, can differ in the test subject's sample from by a decrease of 0.8× control, 0.5× control, 0.2× control to less than 0.001× control and numbers and fractional amounts therebetween, or an increase of 1.2× control, 1.5× control, 4.0×, 6.0× control to >10× control or numbers or fractional amounts therebetween.

Based on the selection of the control and analysis, modification of one or more glycans is detected in the glycomic signature. The glycomic signature is then used as an indication of HIV persistence and/or the developing an HIV-comorbidity, and/or the size of the HIV+ reservoir, as demonstrated in the examples and data figures attached hereto.

In one embodiment, the method identifies the selected glycan is sialic acid or its derivatives and the selected modification as hyposialylation. As shown in the figures and examples below, hypo-sialyation persists in HIV+ individuals despite long-term suppressive ART. The inventors used the glycomic signatures generated in the examples to determine that HIV-infection is associated with persistent loss of sialylated glycans in total IgG glycome. The data provided herein identifies that HIV infection is also associated with persistent alterations in the IgG glycome including decreased levels of di-sialylated glycans, which is associated with a lower anti-inflammatory activity. Thus, this hyposialylation modification is indicative of HIV persistence, poor immune reconstitution after ART and can indicate the potential for certain inflammatory comorbidities.

In still another embodiment, the selected glycan is fucose and the modification is hyper-fucosylation. The data provided herein identifies that HIV infection is also associated with persistent alterations in the IgG glycome including increased levels of fucosylated glycans, which is associated with lower antibody-dependent cell-mediated cytotoxicity (ADCC). In one embodiment a glycomic profile indicative of persistent HIV and comorbidities comprises hypofucosylation. In another embodiment, the profile includes both hypofucosylation and hyposialylation in the IgG glycome.

Finally, levels of certain circulating anti-inflammatory glycans are associated with higher levels of CD4 T cells and lower levels of T cell activation. Our data provide evidence that these glycomic alterations are associated with levels of HIV persistence in the setting of ART suppression. Certain glycomic traits, e.g., IgG galactosylation, in isolated IgG glycomes correlate with HIV persistence during ART. In another embodiment of the method, the selected modification is hypo-galactosylation, which is indicative of development a large reservoir of HIV and thus HIV persistence. As discussed herein, and shown particularly in the figures and supported by the data, various glycans can form glycomic signatures indicative of HIV persistence and co-morbidity.

In yet another aspect, a method for treating an HIV-infected subject comprises modifying or normalizing the level of a selected dysregulated glycan structure in the subject's glycome. In one embodiment, these treatment methods can be combined with the steps involved in the methods of determining HIV persistence and likelihood of comorbidity described above. In another embodiment, the treatment methods can stand alone.

In one embodiment, the treatment methods comprise increasing the level of a selected glycan, e.g., sialic acid or galactose, to that of an uninfected control or an Immune Responder control. In yet a further embodiment, the treatment methods comprise decreasing the level of a selected glycan, e.g., fucose, to that of an uninfected control, an Immune Responder control, or a control negative for an HIV comorbidity.

Thus, one embodiment of the methods of treatment of HIV+ subjects involves increasing the level of sialic acid or sialylated proteins in the subject. Another embodiment of the methods of treatment of HIV+ subjects involves increasing the level of galactose or galactosylated proteins in the subject. In still another embodiment, the methods of treatment of HIV+ subjects involve decreasing the level of fucose or fucosylated proteins in the subject.

These treatment methods are designed to prevents the early development of inflammation- and inflammation-associated diseases in HIV+ individuals. In another embodiment, these methods are designed to reduce the size of the HIV reservoir. In yet a further embodiment, these methods are designed to increase immune reconstitution, and decrease adverse immune activation and dysregulation in HIV+ individuals.

In certain embodiments of the treat methods, the manipulation (increase or decrease) of the amount of the selected glycan in vivo occurs during ART treatment. In certain embodiments of the treatment methods, the manipulation (increase or decrease) of the amount of the selected glycan in vivo occurs subsequent to treatment of the subject with ART. In still other embodiments the methods are directed to occur between ART treatments.

In performing the treatment methods, many therapeutic agents can be administered to the subject to modify (increase or decrease) the selected glycan. Typical therapeutic agents useful to accomplish the increase of a selected glycan (e.g., sialic acid) involve the in vivo administration of the selected glycan in a suitable pharmaceutical carrier or excipient. In one embodiment, the agent is a carrier or nanoparticle coated with the glycan. As demonstrated in the examples and figures herein, sialic acid can be conjugated with a poly (lactic-co-glycolic acid) (PLGA) nanoparticle. Still other known nanoparticles can be used for the same purpose.

The methods of treatment may also employ as a therapeutic agent a conjugate formed of a targeting moiety and the selected glycan (s) to deliver the selected glycan to only particular locations in the subject's body, e.g., cancer cells, specific normal cell types or a tissue location. Still additional mechanisms to increase a glycan that has demonstrated underexpression associated with persistent HIV or a comorbidity is employing a compound or precursor in the glycan biosynthetic pathway or derivative thereof.

In still another treatment method in which the manipulation involves decreasing a selected glycan such as fucosylated structures noted to be overexpressed in HIV persistence or in a co-morbidity involves use of an inhibitor of the glycan or its precursor in the glycan biosynthetic pathway or derivative thereof. Similarly, glycosylation inhibitors or deglycosylation enzymes may be provided to the subject.

Other therapeutic agents are those that are useful in manipulating glycosyltransferases expression. Similarly, antibodies conjugated to glycan-manipulating molecules may be used. Certain antibodies or other targeting proteins can be conjugated with the selected glycan ex vivo and administered to the subject.

Other methods to increase or decrease or correct levels of the selected glycan may involve genetic manipulation of the host glycome, such as by techniques such as CRISPR methods, recombinant viruses that either increase or reduce expression of a target, etc.

Additional methods of treatment are indirect and involve treating cells, e.g., PBMCs, from the subject ex vivo with glycan coated nanoparticles to correct a deficiency in the glycan level.

Further embodiments of the methods of treatment or identification of HIV persistence and co-morbidities are demonstrated in the examples and figures herein.

As described in the Examples below, we examined relationships between the glycomes of plasma and total immunoglobulin G (IgG) and nucleic-acid measures of HIV persistence during and after ART. We applied advanced glycomic technologies to samples (plasma and cells) from INRs and IRs as well as longitudinal samples from a well-characterized cohort of HIV-infected individuals started art with a CD4+ T cell count below 100 cells/mm³. First, we compared glycomes of total plasma and isolated immunoglobulin G (IgG) from HIV+ ART-suppressed, HIV+ viremic, and HIV-negative individuals. Second, in ART-suppressed individuals, we examined the associations between glycomes and 1) levels of cell-associated HIV DNA and RNA in PBMCs and isolated CD4+ T cells, 2) CD4 count and CD4%, and 3) expression of CD4+ T-cell activation markers. HIV infection is associated with persistent alterations in the IgG glycome including decreased levels of di-sialylated glycans, which is associated with a lower anti-inflammatory activity, and increased levels of fucosylated glycans, which is associated with lower antibody-dependent cell-mediated cytotoxicity (ADCC). We also show that levels of certain mono- and di-galactosylated nonfucosylated glycomic traits (A2G1, A2G2, and A2BG2), which have been reported to be associated with higher

ADCC and higher anti-inflammatory activities, exhibit significant negative correlations with levels of cell-associated total HIV DNA and HIV RNA in ART-suppressed individuals. Finally, levels of certain circulating anti-inflammatory glycans are associated with higher levels of CD4 T cells and lower levels of T cell activation. Our data provide evidence that these glycomic alterations are associated with levels of HIV persistence in the setting of ART suppression. For the first time, we report novel plasma and IgG glycomic alterations that are associated with suppressed HIV infection as well as with CD4 T-cell lymphocyte levels of HIV DNA and RNA during suppressive antiretroviral therapy.

Given the recent paradigm on the critical roles of plasma and IgG glycosylation in modulating several immunological functions and the documented role of anti-HIV-1 antibody glycosylation in controlling HIV infection, we hypothesized that altered plasma and IgG glycosylation during HIV infection plays an important role in determining HIV reservoir size during suppressive therapy. Our data revealed that certain IgG and plasma glycomic alterations associate with HIV infection, some of them are persistent despite suppressive ART, while some are reversible. Induction of agalactosylated glycan in total IgG glycome was previously associated with HIV infection and inflammation and was confirmed in our study^{58,59}. In addition, we revealed certain glycomic alterations that have been associated with inflammation (a loss of disialylated glycans and an increase of fucosylated glycans) and reduction of ADCC activity (an increase of fucosylated glycans) ¹³⁻¹⁵, persist during suppressed HIV infection. These glycomic dysregulations may underlie chronic inflammation and immunological dysfunction during suppressed HIV infection thus contributing to the pathogenesis of HIV-associated comorbidities.

Our data also revealed that a few select glycomic traits, mono- and di-galactosylated glycan structures with no sialic acid or core fucose, glycans structures, in both total plasma and isolated IgG exhibit significant negative correlations with levels of HIV DNA and cell-associated HIV RNA in unfractionated PBMCs and isolated CD4+ T cells. The biological roles of galactosylated IgG have been characterized, providing key insights into the mechanisms underlying our observations. Of interest, IgG galactosylation drives better ADCC, ADCP, and CDC in monoclonal antibodies. Antibody galactosylation has also been associated with an anti-inflammatory activity, by promoting the association between FcyRIIB (CD32b) and dectin-1¹⁶. While antibody glycosylation is programmed in an antigen-specific manner, future data is expected to demonstrate that higher galactosylation of anti-HIV-1 antibodies correlate with better ADCC, ADCP, and/or CDC activity against HIV-infected cells during suppressive therapy. Concurrently, galactosylated antibodies are expected to promote an anti-inflammatory state decreasing immune activation and chronic inflammation during ART. Lower activation can contribute to a reduction of persistent HIV reservoir by preventing the potential HIV on-going replication in tissues during ART. We theorize that in vitro and animal model experiments demonstrate the direct effects of these glycan structures on HIV persistence. We theorize that host cellular and circulating glycomic features refine current known immune associations with persistent HIV, including associations with surface expression of activation markers and immune negative checkpoints (PD-1, TIGIT, and LAG-3), and expression of select anti-HIV-1 host restriction factors. A better understanding of host and viral factors driving the formation and maintenance of the latent HIV reservoir aids the development of effective curative strategies.

Additionally, the data demonstrates the relationship between the plasma and IgG glycomes and cellular activation in the setting of ART. Cell-based measurements of viral persistence are consistently associated with markers of immune activation. In line with our hypothesis, levels of glycans known to promote anti-inflammatory activity (sialylated and galactosylated glycan structures) in the plasma and isolated IgG exhibited positive correlations with CD4 count and CD4% and significant negative correlations with the percentage of CD4+ T cells expressing markers of T-cell activation. Our data, therefore, suggest that the documented anti-inflammatory roles of certain circulating glycans may impact immune activation and chronic inflammation during therapy. These anti-inflammatory features of circulating glycans are useful in interventions to attenuate HIV-associated inflammation and immune activation during suppressed ART.

Genetics, diet, age, and gender, impact antibody glycosylation. While we did not detect an influence of age on our main results, studies with a larger sample size can determine the full extent of the association between circulating glycans and HIV persistence adjusting for all factors that can influence antibody glycosylation. Second, as antibody glycosylation is programed in an antigen-specific manner, each anti-HIV specific antibody glycosylation is likely to have an impact on HIV persistence. Data obtained from blood is expected to be supported by studies involving the enrichment of the HIV reservoir in tissues. Such studies to analyze the role of circulating glycans in maintaining HIV persistence in tissues can be performed in an analogous manner. Lastly, our data describe cross-sectional samples from chronically-infected adults. It is anticipated that analysis of longitudinal changes, acute infection, and both pediatric and older aged cohorts will provide additional data. In addition, in this study, we used nucleic acid-based measures of HIV reservoir size. Currently, there is no gold standard method to measure the size of HIV reservoir. The quantitative viral outgrowth assay underestimates the size of HIV reservoir, while the nucleic acid-based measurements may overestimate it, as only a portion of latently infected cells is able to reactivate and produce replication-competent virus. However, recent reports indicated that levels of HIV DNA and RNA during ART are predictors of viral rebound upon therapy interruption. Therefore, these nucleic acid measures have a biological value.

In summary, our data represent the first proof-of-concept evidence of plasma and IgG glycomic alterations in vivo that modulate HIV persistence in the setting of suppression. These data support the role of antibody glycosylation in modulating certain immune functions and suggests that these glycomic alterations can act as biomarkers of HIV persistence. The methods and compositions described herein for novel glycan-based interventions for treating HIV infection or preventing or ameliorating inflammation-associated comorbidities.

### EXAMPLES

The invention is now described with reference to the following examples. These examples are provided for illustration only. The compositions, experimental protocols and methods disclosed and/or claimed herein can be made and executed without undue experimentation in light of the present disclosure. The protocols and methods described in the examples are not considered to be limitations on the scope of the claimed invention. Rather this specification should be construed to encompass any and all variations that become evident as a result of the teaching provided herein. One of skill in the art will understand that changes or variations can be made in the disclosed embodiments of the examples, and expected similar results can be obtained. For example, the substitutions of reagents that are chemically or physiologically related for the reagents described herein are anticipated to produce the same or similar results. All such similar substitutes and modifications are apparent to those skilled in the art and fall within the scope of the invention.

### EXAMPLE 1: HIV-Associated Hypo-Sialylation Is Linked to Poor Immune Reconstitution and Chronic Inflammation after ART In Vivo

To determine whether HIV-associated hypo-sialylation is linked to poor immune reconstitution and chronic inflammation after ART in vivo, we use several advanced glycomic technologies to compare glycomic alterations in plasma samples from 50 INR individuals and 50 IR individuals. We correlate these alterations with CD4 counts, markers of immune activation, and HIV persistence.

Clinical cohorts: We have access to robust numbers of well characterized HIV+ individuals via the Penn Center for AIDS research (CFAR) clinical core cohort, with stored plasma and cell samples from over 3000 HIV+patients, including samples from over 100 INR). The samples include 50 samples from IRs (CD4 count>500 cells/μl ≥2 years post-suppressive ART) and INRs (CD4 count<350 cells/μl ≥2 years post-suppressive ART). IR controls are selected via propensity score matching to INR individuals based on age, nadir CD4+ T cell count, and duration of ART. In addition, access to plasma and PBMC samples from another study (conducted in S. Africa) within the CADIRIS cohort, as described in the parent studies⁴⁴⁻⁴⁷.

All patients were ART-naive with a CD4 count <100 cells/mm³ before starting ART (efavirenz, tenofovir, and emtricitabine). Plasma and PBMCs were isolated at 0 (pre-ART), 12, and 24 week post-ART. CD4 count, viral load, T cell and monocyte activation, plasma levels of sCD14 and sCD163 are available from all samples.

We use plasma samples from the abovementioned sources to identify cell-free glycomic signatures (in total plasma and isolated IgG). Isolated proteins are labeled with Cy5 and hybridized to a lectin microarray. The lectin array employs a representative panel of 96 immobilized lectins with known glycan structures binding specificity:⁴⁸⁻⁵⁵ The resulting lectin chip is scanned for fluorescence intensity on each lectin-coated spot using an evanescent-field fluorescence scanner GLYCOSTATION Reader. We use ultra-performance liquid chromatography (UPLC) for additional screening of N-linked glycans. N-glycans from isolated proteins are released with PNGase F, labeled with 2-aminobenzamide, and excess reagents are removed using hydrophilic interaction liquid chromatography solid phase extraction (HILIC-SPE).^{56,57} Fluorescently labeled and purified N-glycans are separated by HILIC-UPLC using an Acquity UPLC instrument as previously described.^{56,57} N-glycans are separated into distinct peaks and expressed as a percentage of total integrated area.

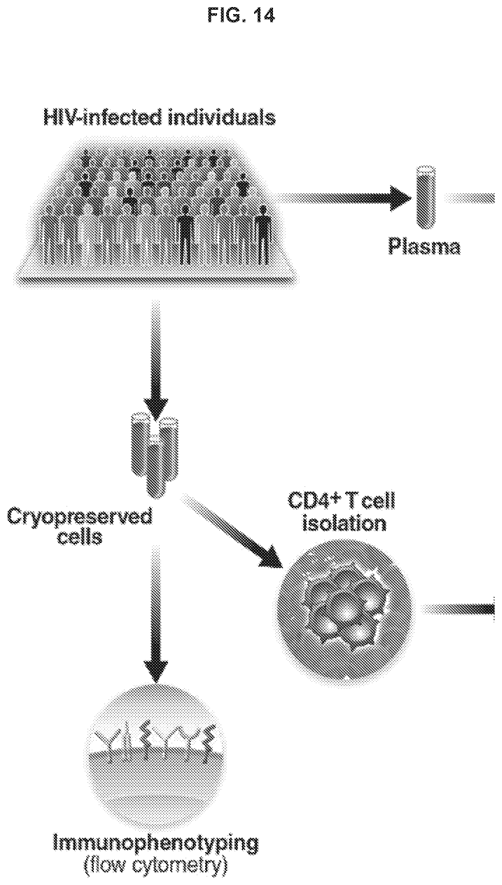

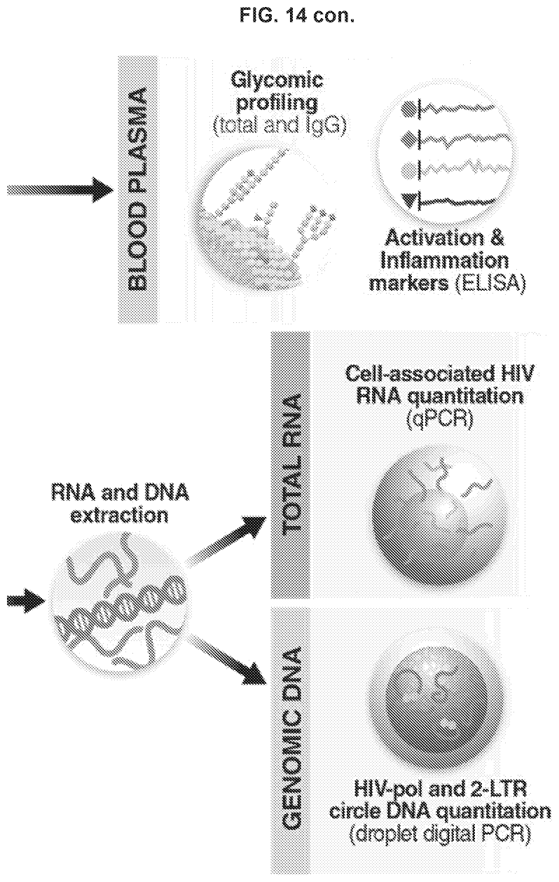

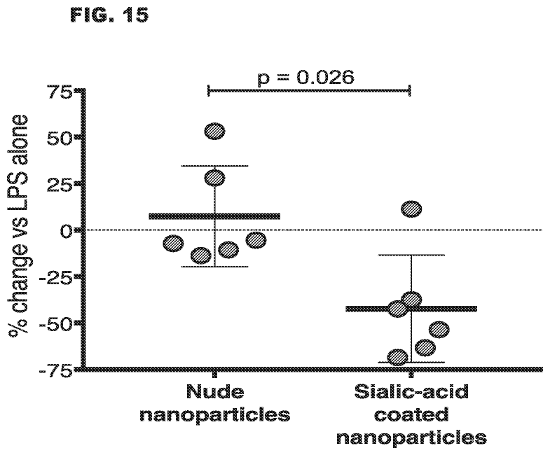

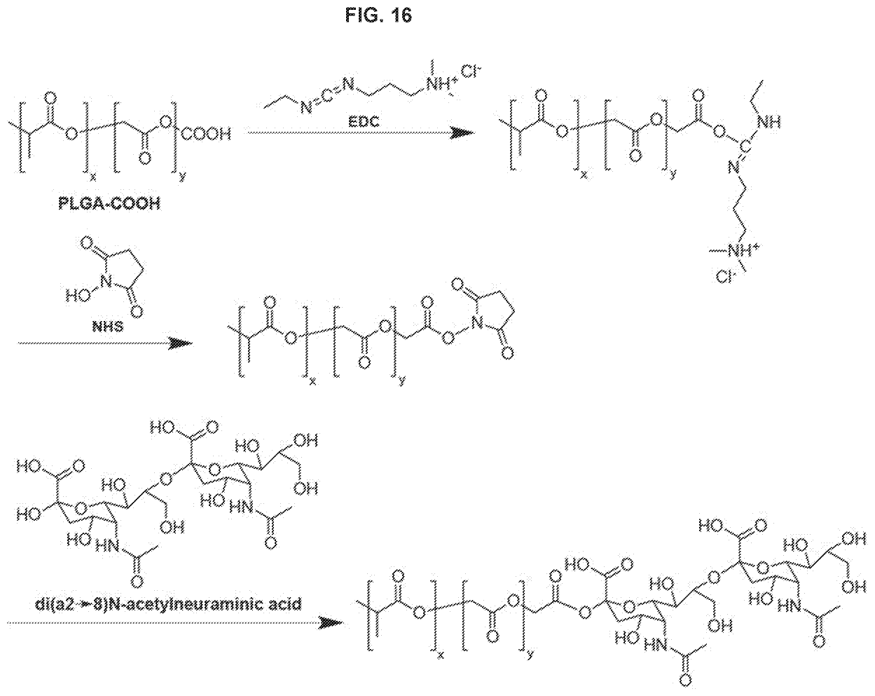

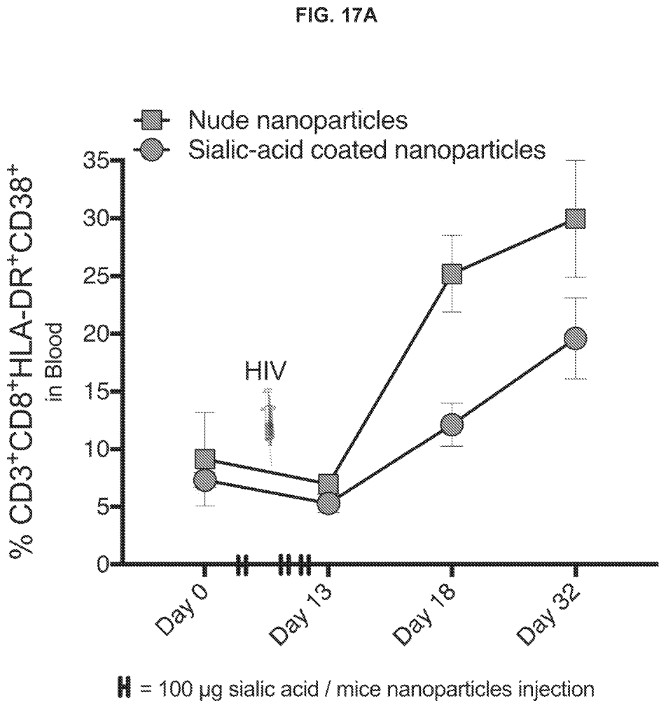

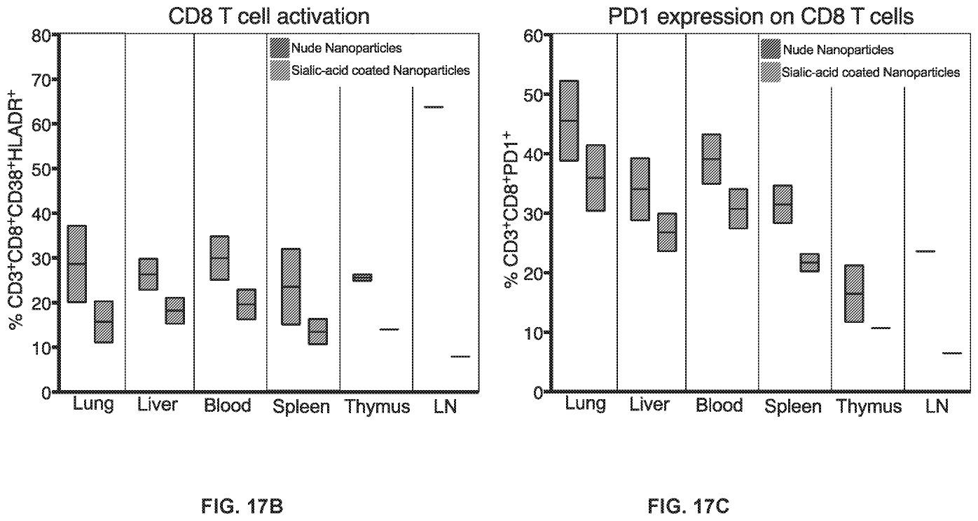

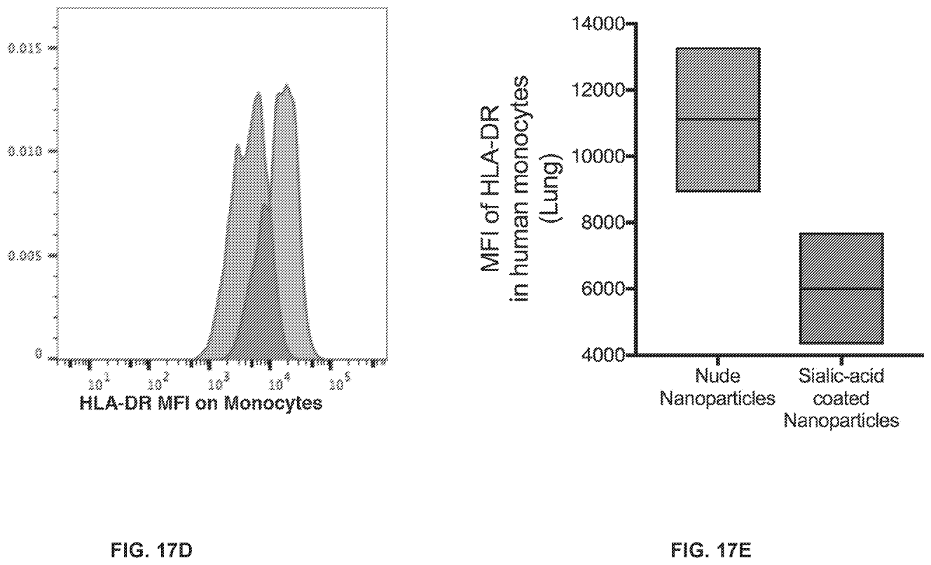

Our preliminary measures showed that 250 μl of plasma is sufficient for glycomic analyses. Glycomic profiles from these samples are subjected to the workflow shown in FIG. 14. First, to determine if INRs exhibit different levels of HIV-associated glycomic dysregulations compared to IRs, we compare glycomic signatures between INRs and IRs (and use HIV-negative samples as controls). Our preliminary data identified hypo-sialylation to be associated with HIV infection even with suppressive art. At a minimum, we evaluate hypo-sialylated glycan classes. However, we expect that additional HIV+-associated glycomic dysregulations will be identified. These findings demonstrate that HIV, even under suppressive long-term ART, alters the host glycome.

To determine if immune reconstitution correlates with HIV-associated glycomic dysregulations, we correlate CD4+T cell counts with glycomic signatures using the cross-sectional INRs vs. IRs samples. We also correlate CD4 reconstitution with glycomic signatures using the longitudinal CADIRIS samples.

To determine if plasma markers of microbial translocation and inflammation correlate with levels of HIV-associated glycomic dysregulations, we use the plasma samples to measure markers of microbial translocation and inflammation, including immunoglobulin M endotoxin core (EndoCAb), intestinal fatty-acid-binding protein (I-FABP), soluble CD14 (sCD14), and interleukin 6.

To determine if levels of monocyte activation correlate with levels of HIV-associated glycomic dysregulations, we profile monocytes activation from the same samples above using the following protocol: PBMCs are phenotyped based on CD14 and CD16 expression and split into three subsets: classical (CD14++CD16−), activated intermediate (CD14++CD16+) and non-classical (CD14low/+CD16++) gated on the CD11B+HLA-DR+PMBC live cell population that excludes T cells, B cells and NK cells (CD3-CD19-CD14-CD2O-CD56-) and dead cells (aqua amine reactive dye).

To determine if cell measures of HIV reservoir size correlate with levels of HIV-associated glycomic dysregulations, we isolate CD4+T cells from PBMCs. Total RNA and DNA are extracted and used to measure the levels of cell-associated HIV RNA and total HIV DNA using RT-qPCR and ddPCR.

Analytical methods: Glycan structures are evaluated and compared between the control group and each of the two HIV+groups (IRs and INRs), and between the INRs and IRs. A 2-fold difference in means is considered biologically meaningful. Due to multiple testing, a false discovery rate (FDR) of 0.05 using a two-sided two-sample t-test will be applied. Among 96 glycan structures, we expect 10% will have at least 2-fold difference in means between compared groups; the statistical significance level is thus set as 0.005 for each test of two groups comparison.

From these data, we expect that the maximum coefficient of variation for the measurement will not be greater than 0.8. With all these conditions, to have 90% power, 50 individuals in each group is sufficient. Spearman's rank test is applied to serially to evaluate the associations between glycan structures and CD4 counts, levels of plasma markers of inflammation, monocyte cell-surface activation, and cellular measures of HIV persistence in each of the groups separately. Power analysis indicated that a sample size of 50 is sufficient to achieve 90% power to detect a correlation of r=0.63 using a two-sided hypothesis test with a significance level of 0.005 assuming that 10% of 96 glycan structures would have r>=0.63 and FDR is 5%.

Projected results: We expect that INRs have higher levels of hypo-sialylation compared to IRs. We expect that levels of certain glycan structures correlate significantly with CD4 counts, markers of microbial translocation, immune activation, chronic inflammation, and HIV reservoir size, thereby defining glycome signatures for HIV infection and HIV treatment status. Importantly, we generate the first-of-its-kind dataset on in vivo circulating glycomes across HIV treatment groups, and their association with markers of chronic inflammation and HIV reservoir size. This dataset is useful to guide the development of novel therapeutic agents for chronic inflammation, which can prevent the development of inflammation-associated HIV comorbidities by manipulating levels of sialic acid and other glycan structures.

To identify a differentiating glycan structure, we use other glycomic technologies such as mass spectrometry or nuclear magnetic resonance. We also analyze the expression of 424 glycan-related genes in the isolated cells using RNAseq, and then correlate the expression levels of those glycan-related genes with levels of HIV persistence and chronic inflammation.

### EXAMPLE 2: Elevation of Sialic Acid Levels Can Prevent Monocyte Activation and Cell Apoptosis Ex Vivo.

This study uses sialic acid coated nanoparticles, which abrogate monocyte activation/inflammation in our preliminary in vitro work, to provide evidence that increasing the amount of sialic acid, by treating primary PBMCs from INRs and IRs with sialic-acid coated nanoparticles, reduces monocyte activation and caspase-dependent CD4 apoptosis.

We examine the link between hypo-sialylation and immune reconstitution by assessing the glycomic profiles of longitudinal plasma samples from a well-characterized HIV cohort who started ART with a CD4 count<100 cells/mm³.

Our data show that hypo-sialylation might be an irreversible consequence of HIV infection despite suppressive art for many years. Hypo-sialylation has been associated with chronic inflammation, immune activation, and cell death⁸⁻¹⁶. Sialic acid-coated nanoparticles have been used to reduce inflammation ex vivo and in mouse models. We test if treatment with sialic acid-coated nanoparticles attenuates LPS-induced production of proinflammatory cytokines from PBMCs isolated from INRs and IRs. Monocyte activation and migration have been associated with the development of HIV-associated comorbidities and INR phenotype.^{19,20} In addition, we test if these molecules can prevent CD4+ T cell death upon FasL, TRAIL, or TNF-α stimulation.

PBMCs from 10 HIV+ INRs, 10 HIV+ IRs, and HIV-negative controls are stimulated with either LPS (100 ng/ml) or media alone for 6 hours in the presence or absence of sialic acid-coated nanoparticles (University of Pennsylvania Department of Bioengineering). Cells are stained to define monocyte populations based on CD14, and CD16 expression and culture supernatant levels of TNF-α, IL-6 and IL-1α are measured by ELISA.

We assess monocyte mobilization in an established chemotaxis assay, which uses a Boyden chamber with a porous membrane devoid of endothelial cells. Under MCP-1 pressure, we examine monocyte migration under the same conditions as above. Data is presented as a migratory factor by dividing the number of migrated monocytes by the total number of monocytes added to the insert and expressing that ratio as a percentage.

PBMCs from 10 HIV+ INRs, 10 HIV+ IRs, and HIV-negative controls, are cultured in the presence of absence of sialic acid nanoparticles. Cells are stimulated with recombinant FasL, TRAIL, or TNF-α. CD4+ T cell death is assessed by flow cytometry using BV605-labeled Annexin v, Sytox-Red, and antibodies for human CD3 and CD4. Caspase activity is measured using caspase activity assays (ThermoFisher).

Statistical significance is calculated by comparing each treatment group to the controls using paired t-tests or the Wilcoxon signed-rank test. A sample size of 10 provides 80% power to detect a mean of 2% in paired difference with an estimated 2% standard deviation of the paired differences at an alpha level of 0.05 using a two-tailed paired t-test. Our ex vivo studies establish the ability of these molecules to serve as a therapeutic candidate to enhance immune reconstitution and prevent the development of HIV-associated comorbidities and advance this compound towards clinical testing, after testing it in humanized mouse models and non-human primates.

These studies untangle the association between HIV and the host immune environment after ART, taking advantage of recent advances in glycan-based technologies. The results of these examples create a new paradigm for novel glycan-based interventions that enhance immune reconstitution and prevent HIV-associated comorbidities in the INR population.

The physiological processes underlying poor immune reconstitution after ART remain poorly understood and likely involve multifactorial mechanisms. Many factors have been theorized to cause poor CD4+ T cell reconstitution after successful ART. These include problems with CD4+ T cell production, destruction of CD4+ T cells by immune activation, dysfunctional regulatory T cells (Treg) cells, and loss of lymph node architecture.⁴ Other problems include possible on-going HIV production, Cytomegalo-virus (CMV) infection, loss of regulatory T cells (Tregs), microbial translocation, and other factors.

Inflammation has been associated with aberrant glycosylation patterns, in particular, loss of sialic acid. All living cells assemble a diverse repertoire of glycan (carbohydrate) structures on their surface.²³ Recent advances in glycobiology show that host glycosylation and glycan-lectin signaling play critical roles in immune responses⁵ and in cell-cell⁶ and cell-pathogen interactions.⁷ Altered glycosylation is documented in many diseases including cancer, autoimmune²⁵, and infectious diseases⁵⁸. Altered serum cell-free circulating glycan structures have been identified as biomarkers for cancer²⁴ and in various cellular processes.²⁵

Glycans can enter the circulation from tissues through active secretion or leakage, but antibody glycosylation also plays a critical role in the impact of circulating glycans on the immune system²⁶.

These include the cell-free glycoproteins, which can enter the circulation from tissues through active secretion or leakage, and the glycans on antibodies/IgGs.²⁶ IgG-associated glycans are of interest because these regulate the binding of IgG to its various receptors, which in turn define the IgG pro- and anti-inflammatory responses. For example, a reduction in IgG sialylation is known to increase the pro-inflammatory function of IgG.¹²⁻¹⁶ Glycosylation allows antibodies to interact with classical and non-classical Fc receptors, which defines the antibody functionality, e.g., in inducing antibody-dependent cell-mediated cytotoxicity (ADCC) and anti-inflammatory activities.²⁷⁻³⁰ Antibody sialylation and galactosylation are linked to anti-inflammatory responses.¹³⁻¹⁵

One model of how altered glycosylation influences immune response stems from the observation that binding of sialic acid-containing glycans to sialic acid-binding proteins (siglecs) on the surface of monocytes and macrophages initiates an inhibitory signal that leads to an anti-inflammatory response, through inhibition of TLR4 signal transduction.³¹⁻³⁴ Thus, loss of sialic acid (hypo-sialylation), as in HIV infection, induces inflammation because it reduces the opportunity for sialic acid/SIGLECS anti-inflammatory binding on monocytes and macrophages. Consistently, activation of monocytes has been associated with the development of HIV-associated comorbidities³⁵⁻⁴¹ and caspase-mediated cell death is strongly modulated by sialic acid.

Sialylated glycans are reduced in the plasma of HIV+ individuals. We have been investigating glycomic alterations in HIV+ individuals and HIV− controls. Using a new lectin microarray technology, we found that levels of sialylated glycans were reduced in plasma of HIV viremic and art-suppressed individuals compared to HIV− individuals (FDE<0.01) (FIG. (A-9B). This suggests that systemic, HIV-associated hypo-sialylation persists despite long-term suppressive art. Our preliminary work also shows that levels of a particular class of hypo-sialylated N-linked oligosaccharides (unsialylated β (1,4) Gal GlcNAc) is positively correlated with the degree of neurological impairment in HIV+ individuals on suppressive art (data not shown).

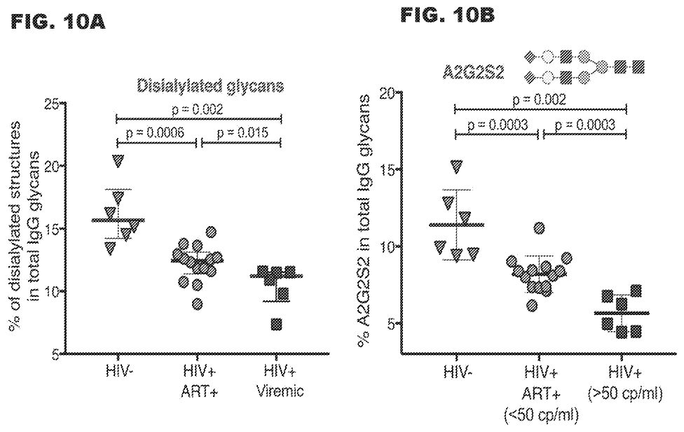

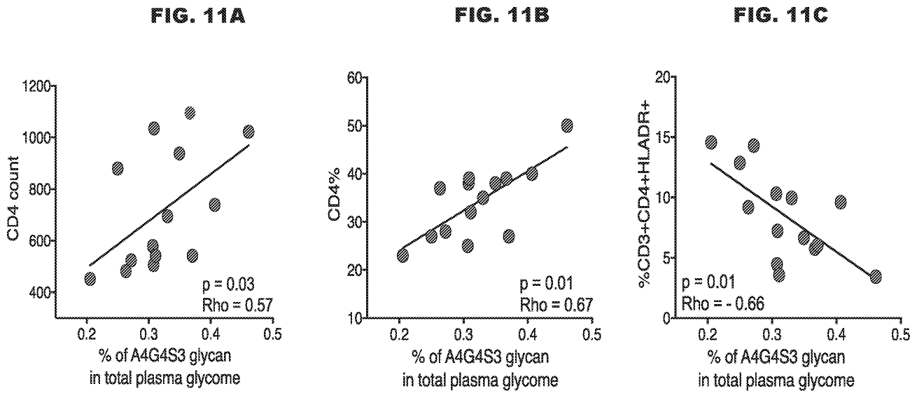

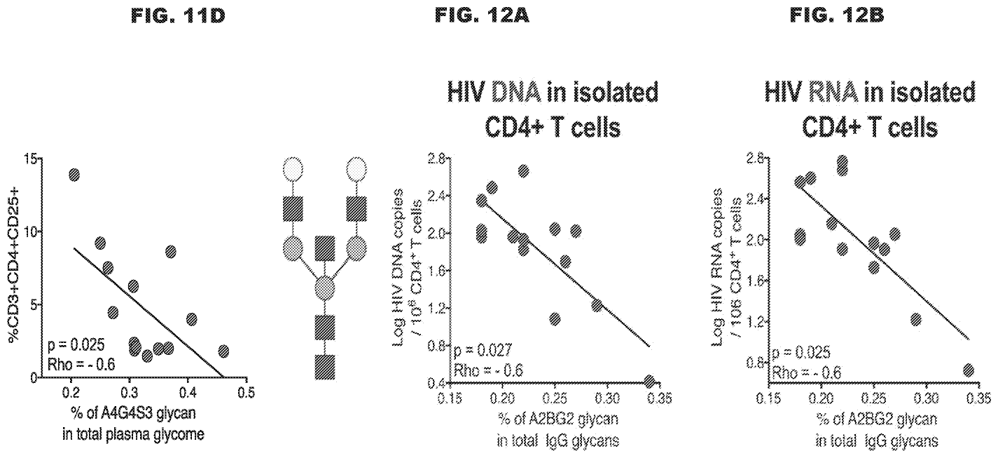

In addition, using a different cohort and a different glycomic profiling method (UPLC), we found that HIV infection is associated with persistent alterations in the IgG glycome, including decreased levels of di-sialylated glycans (in particular, the A2G2S2 glycan trait), which is associated with lower anti-inflammatory activity¹³⁻¹⁶ (FIGS. 10A and 10B). We also found that levels of specific plasma anti-inflammatory glycans (A4G4S3, known to be associated with anti-inflammatory activities¹³⁻¹⁶) are associated with higher levels of CD4 T cells (FIG. 11A-11B) and lower levels of T cell activation (FIG. 11C-11D). Furthermore, levels of a glycomic trait (A2BG2) that has been associated with anti-inflammatory effects and higher ADCC activity correlate negatively with levels of CD4+ T cell-associated HIV DNA and RNA during suppressive art (FIG. 12A-12B).

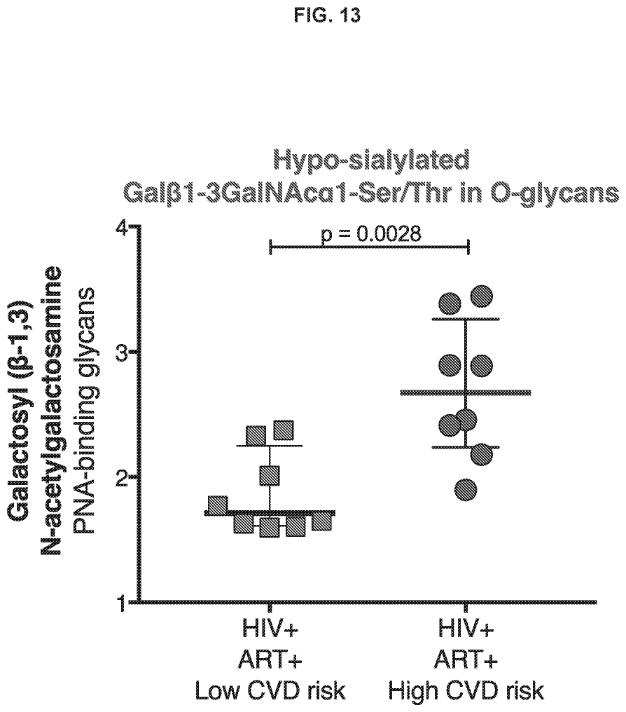

Administration of sialic acid, via sialic acid coated nanoparticles, abrogates inflammation. Sialic acid coated nanoparticles boost anti-inflammatory response in culture, and improve survival in mouse models of disease associated with chronic inflammation, such as sepsis and lung disease.^{42,43} Our preliminary work found that treating human monocytes with sialic acid coated nanoparticles significantly attenuated LPS-induced secretion of pro-inflammatory TNF-α (p=0.026) (FIG. 13). We hypothesize that particles act by enhancing sialic acid-siglec anti-inflammatory binding systemically. Though promising, these molecules have yet to be tested in any HIV-relevant system.

In summary, our data show that altered glycosylation patterns, in particular, hypo-sialylation, persist even after long-term suppressive art, and suggest that the documented anti-inflammatory roles of certain circulating glycans impact immune activation, immune reconstitution, and chronic inflammation during art. Glycomic alterations are shown to contribute to the pathogenesis of immune reconstitution failure and chronic inflammation in HIV+ individuals after ART.

This data demonstrates the glycomic mechanistic underpinnings of HIV-associated immune dysfunction and inflammation. Therapeutic strategies to remodel glycans, e.g. by using glycan-coated nanoparticles, glycosylation inhibitors, or manipulating glycosyltransferases are thus useful as HIV treatments. This data supports a new paradigm for enhancing immune reconstitution and preventing the development of the inflammation-associated HIV comorbidities. The systemic glycomic alterations associated with chronic inflammation is anticipated to have an impact on other diseases involving inflammation (e.g., cardiovascular, and neurological diseases), immune dysfunction (e.g., cancer), and pathogen infections.

### EXAMPLE 3: Plasma and Immunoglobulin G Galactosylation Associate with HIV Persistence During Antiretroviral Therapy

### Methods and Materials

A. Study Population.

Fresh PBMCs and plasma from HIV-infected ART-suppressed (viral load <20 copies/ml), HIV-infected unsuppressed (viral load >50 copies/ml), and HIV-negative individuals were included in the analysis (Table 1) and obtained from Philadelphia FIGHT and The Wistar Institute. Written informed consent was provided by all patients and donors recruited, and the protocols used were approved by the Institutional Review Boards of the University of Pennsylvania and The Wistar Institute. Plasma RNA viral load, CD4+ T cell counts, and CD4% were measured at all patient visits.

TABLE 1
Clinical Data for Study Participants

| | | CD4 | | | | | | | Approx. |
|---|---|---|---|---|---|---|---|---|---|
| | | count | CD4 | VL | | | Age | Current | yrs on |
| | Donor | cells/mm³ | % | copies/ml | Gender* | Race^{#} | yrs | regime\hat{ } | ART |
| HIV | A | — | — | — | M | AA | 65 | — | — |
| neg | B | — | — | — | M | AA | 58 | — | — |
| | C | — | — | — | M | AA | 48 | — | — |
| | D | — | — | — | F | AA | 61 | — | — |
| | E | — | — | — | F | AA | 33 | — | — |
| | F | — | — | — | F | C | 33 | — | — |
| HIV | G | 524 | 28% | <20 | M | AA | 56 | ABC, | 10 |
| VL | | | | | | | | 3TC, | |
| <50 | | | | | | | | DTG | |
| | H | 739 | 40% | <20 | M | AA | 37 | DTG, | 2.5 |
| | | | | | | | | RPV | |
| | I | 1095 | 39% | <20 | M | AA | 32 | FTC, | ~4 |
| | | | | | | | | RPV, | |
| | | | | | | | | TDF | |
| | J | 1022 | 50% | <20 | M | AA | 53 | EVG/c, | ~5 |
| | | | | | | | | FTC, | |
| | | | | | | | | TDF | |
| | K | 506 | 38% | <20 | M | AA | 23 | EVG/c, | 5 |
| | | | | | | | | FTC, | |
| | | | | | | | | TDF | |
| | L | 452 | 23% | <20 | M | AA | 55 | ABC, | 7.5 |
| | | | | | | | | 3TC, | |
| | | | | | | | | DTG | |
| | M | 482 | 37% | <20 | M | AA | 27 | FTC, | 3 |
| | | | | | | | | RPV, | |
| | | | | | | | | TDF | |
| | M | 542 | 27% | <20 | M | AA | 37 | FTC, | 7.5 |
| | | | | | | | | TDF, | |
| | | | | | | | | DRV/r | |
| | O | 880 | 27% | <20 | M | AA | 49 | ABC, | 4.5 |
| | | | | | | | | 3TC, | |
| | | | | | | | | DTG | |
| | P | 579 | 25% | <20 | M | AA | 30 | ABC, | 1.75 |
| | | | | | | | | 3TC, | |
| | | | | | | | | DTG | |
| | Q | 695 | 35% | <20 | M | AA | 25 | EVG/c, | 2 |
| | | | | | | | | FTC, | |
| | | | | | | | | TDF | |
| | R | 1035 | 39% | <20 | M | C | 57 | FTC, | 6 |
| | | | | | | | | RPV, | |
| | | | | | | | | TDF | |
| | S | 938 | 38% | <20 | M | AA | 29 | ABC, | 6.5 |
| | | | | | | | | 3TC, | |
| | | | | | | | | DTG | |
| | T | 542 | 32% | <20 | M | C | 51 | EFV, | 18 |
| | | | | | | | | TDF, | |
| | | | | | | | | FTC | |
| HIV | U | 357 | 25% | 15,748 | M | AA | 29 | no ART | — |
| VL | V | 595 | 28% | 573 | F | AA | 51 | TDF, | 8 |
| >50 | | | | | | | | FTC, | |
| | | | | | | | | RAL | |
| | W | 337 | 26% | 146 | M | AA | 55 | DRV/c, | 15 |
| | | | | | | | | DTG, | |
| | | | | | | | | 3TC | |
| | X | 234 | 7% | 27,380 | F | AA | 50 | DRVc, | 7 |
| | | | | | | | | FTC, | |
| | | | | | | | | DTG | |
| | Y | 223 | 14% | 19,220 | M | AA | 54 | no ART | — |
| | Z | 357 | 17% | 45,333 | M | AA | 48 | no ART | — |

KEY:
*M = male, F = female
^{#}AA = African American, C = Caucasian
\hat{ }FTC, emtricitabine; TDF, tenofovir; 3TC, lamivudine; EFV, efavirenz; ABC, abacavir; RAL, raltegravir; EVG/c, elvitegravir boosted with cobicistat; DTG, dolutegravir; DRV/r, darunavir boosted with ritonavir; DRV/c, darunavir boosted with cobicistat; RPV, Rilpivirin

B. T-Cell Immunophenotyping.

Markers of T-cell activation (HLA-DR, CD69, and CD25) that have been associated with HIV latency [49-53], were measured using flow cytometry. Briefly, freshly isolated PBMCs were stained with LIVE/DEAD® Fixable Aqua Dead Cell Stain Kit (Invitrogen) and then stained with the following fluorescently-conjugated monoclonal antibodies: CD3 BV421 (clone UCHT1), CD4 PE-Cy7 (clone RPA-T4), CD25 BV605 (clone BC96), HLA-DR FITC (clone L243) from Biolegend. CD69 PE (clone FN50) from BD Biosciences. Stained cells were fixed in PBS with 1% paraformaldehyde, and stored in the dark at 4° C. until acquisition. All phenotyping data were collected on BD LSR II (BD Biosciences). Data were analyzed using FlowJo software (Treestar).

qPCR quantification of cellular HIV-1 DNA and RNA. CD4+ T cells were isolated from PBMCS using EASYSEP Human CD4+ T cell enrichment kit (Stemcell Technologies, Vancouver, British Columbia, Canada). Cellular RNA and DNA from total unfractionated PBMCs and isolated CD4+ T cells were purified using the ALLPREP DNA/RNA kit (Qiagen, Ventura Calif.) as specified by the manufacturer, quantified using a Nanodrop (ND-1000) spectrophotometer and normalized to cell equivalents by qPCR using human genomic TERT for DNA and RPLPO expression for RNA (Life Technologies, Grand Island N.Y.). Total cellular HIV-1 DNA and RNA was quantified with a qPCR TaqMan assay using LTR-specific primers F522-43 (5′ GCC TCA ATA AAG CTT GCC TTG A 3′; HXB2522-543) and R626-43 (5′ GGG CGC CAC TGC TAG AGA 3′; 626-643) coupled with a FAM-BQ probe (5′ CCA GAG TCA CAC AAC AGA CGG GCA CA 3) using the QUANTSTUDIO 6 Flex Real-Time PCR System (Applied Biosystems).

Cell-associated HIV-1 DNA copy number was determined using a reaction volume of 20 μl with 10 μl of 2× TaqMan Universal Master Mix II including UNG (Life Technologies), 4 pmol of each primer, 4 pmol of probe, and 5 μl of DNA. Cycling conditions were 50° C. for 2 min, 95° C. for 10 min, then 60 cycles of 95° C. for 15 s and 59° C. for 1 min. Cell-associated HIV-1 RNA copy number was determined in a reaction volume of 20 μl with 10 μl of 2× TaqMan RNA to Ct 1 Step kit (Life Technologies), 4 pmol of each primer, 4 pmol of probe, 0.5 μl reverse transcriptase, and 5 μl of RNA. Cycling conditions were 48° C. for 20 min, 95° C. for 10 min, then 60 cycles of 95° C. for 15 s and 59° C. for 1 min.

For HIV-1 DNA measurements, external quantitation standards were prepared from pNL4-3 in a background of HIV-1 negative human cellular DNA, calibrated to the Virology Quality Assurance (VQA, NIH Division of AIDS) cellular DNA quantitation standards. For HIV RNA measurements, external quantitation standards were prepared from full-length NL4-3 virion RNA followed by copy number determination using the Abbott RealTime assay (Abbott Diagnostics, Des Plains Ill.) and calibrated to VQA HIV-1 RNA standards. Patient specimens were assayed with up to 800 ng total cellular RNA or DNA in replicate reaction wells and copy number determined by extrapolation against a 7-point standard curve (1-10,000 cps) performed in triplicate.

C. IgG Isolation From Plasma.

Plasma samples have been incubated for 30 minutes at 56° C. to inactivate HIV and then stored at −20° C. prior N-glycan analysis. Total IgG from plasma was isolated using CIM® Protein G 96-well plate (BIA Separations, Ajdovs̆c̆ina, Slovenia), and vacuum manifold (Pall Corporation, Port Washington, N.Y., USA). All steps during the isolation procedure were performed at 380 mm Hg, except for plasma sample application and IgG elution (around 200 mm Hg). All buffers were filtered through 0.2 μm PES filters (Nalgene Thermo Fischer Scientific, Waltham, Me., USA). Plasma samples (100 μL) were centrifuged for 3 minutes at 12 100 g, pipetted to the previously designated wells of a collection plate and diluted with 1× PBS, pH 7.4 in the ratio 1:7. All diluted plasma samples were filtered through 0.45 μm and 0.2 μm ACROPREP GHP filter plates (Pall Corporation) using vacuum manifold (around 380 mm Hg) and immediately applied to preconditioned Protein G plate as was previously described⁵⁷. Protein G plate was then washed, and IgG eluted with 0.1 mol L-1 formic acid and immediately neutralized with ammonium bicarbonate to pH 7,0. Protein G plate was regenerated and stored at 4° C. The volume of 300 μL from each elution fraction was dried in a vacuum centrifuge with vacuum concentrator Savant SC210A, refrigerated vapor trap Savant RVT400 and vacuum pump OFP400 (Thermo Scientific) for subsequent N-glycan analysis.

D. N-Glycan Release, Labeling, and Analysis by Ultra Performance Liquid Chromatography (UPLC).

N-glycans from isolated IgG and plasma samples (10 μL) were released with PNGase F, labeled with 2-aminobenzamide and excess of regents removed by clean-up using hydrophilic interaction liquid chromatography solid phase extraction (HILIC-SPE), as previously described^{56, 57}. Eluates were stored at −20° C. until the ultra-performance liquid chromatography (UPLC) analysis. Fluorescently labeled and purified N-glycans were separated by HILIC-UPLC using Acquity UPLC instrument (Waters, Milford, Mass., USA) as previously described ^{56,57}. IgG N-glycan samples were all separated into 24 peaks²⁶ and total plasma N-glycans into 39 peaks [56]. The amount of N-glycans in each chromatographic peak was expressed as a percentage of total integrated area (% Area). From these directly measured IgG and total plasma N-glycans, additional glycan traits have been derived to assess changes in the amount of glycan classes that represent structurally similar glycan species.

TABLE 2
Directly measured and derived IgG N-glycan traits.

| | Trait | | Formula |
|---|---|---|---|
| | | % of Specified Glycan in | |
| | | Total IgG Glycans | |
| Directly | GP1 | FA1 glycan | GP1/GP * 100 |
| measured | GP2 | A2 glycan | GP2/GP * 100 |
| IgG N- | GP4 | FA2 glycan | GP4/GP * 100 |
| glycan | GP5 | M5 glycan | GP5/GP * 100 |
| traits | GP6 | FA2B glycan | GP6/GP * 100 |
| | GP7 | A2G1 glycan | GP7/GP * 100 |
| | GP8 | FA2[6]G1 glycan | GP8/GP * 100 |
| | GP9 | FA2[3]G1 glycan | GP9/GP * 100 |
| | GP10 | FA2[6]BG1 glycan | GP10/GP * 100 |
| | GP11 | FA2[3]BG1 glycan | GP11/GP * 100 |
| | GP12 | A2G2 glycan | GP12/GP * 100 |
| | GP13 | A2BG2 glycan | GP13/GP * 100 |
| | GP14 | FA2G2 glycan | GP14/GP * 100 |
| | GP15 | FA2BG2 glycan | GP15/GP * 100 |
| | GP16 | FA2G1S1 glycan | GP16/GP * 100 |
| | GP17 | A2G2S1 glycan | GP17/GP * 100 |
| | GP18 | FA2G2S1 glycan | GP18/GP * 100 |
| | GP19 | FA2BG2S1 glycan | GP19/GP * 100 |
| | GP20 | FA2FG2S1 glycan | GP20/GP * 100 |
| | GP21 | A2G2S2 glycan | GP21/GP * 100 |
| | GP22 | A2BG2S2 glycan | GP22/GP * 100 |
| | GP23 | FA2G2S2 glycan | GP23/GP * 100 |
| | GP24 | FA2BG2S2 glycan | GP24/GP * 100 |
| | | % of Specified Structure | |
| | | in Total IgG Glycans | |
| Derived | G0 | agalactosylated structures | (GP1 + GP2 + GP3 + |
| IgG N- | | | GP4 + GP6)/GP *100 |
| glycan | G1 | monogalactosylated | (GP7 + GP8 + GP9 + |
| traits | | structures | GP10 + GP11)/GP *100 |
| | G2 | digalactosylated structures | (GP12 + GP13 + GP14 + |
| | | | GP15)/GP *100 |
| | F | all fucosylated structures | (GP1 + GP4 + GP6 + |
| | | (+/− bisecting GlcNAc) | GP8 + GP9 + GP10 + |
| | | | GP11 + GP14 + GP15 + |
| | | | GP16 + GP18 + GP19 + |
| | | | GP20 + GP23 + |
| | | | GP24)/GP *100 |
| | B | structures with bisecting | (GP3 + GP6 + GP10 + |
| | | GlcNAc | GP11 + GP13 + GP15 + |
| | | | GP19 + GP22 + |
| | | | GP24)/GP * 100 |
| | S | sialylated structures | (GP16 + GP17 + GP18 + |
| | | | GP19 + GP20 + GP21 + |
| | | | GP22 + GP23 + |
| | | | GP24)/GP * 100 |
| | S1 | monosialylated structures | (GP16 + GP17 + GP18 + |
| | | | GP19 + GP20)/GP * 100 |
| | S2 | disialylated structures | (GP21 + GP22 + GP23 + |
| | | | GP24)/GP * 100 |

TABLE 3
Directly measured and derived total plasma N-glycan traits.

| | Trait | | Formula |
|---|---|---|---|
| | | % of Specified Glycan in | |
| | | Total Plasma Glycans | |
| Directly | GP1 | FA2 glycan | GP1/GP * 100 |
| measured | GP2 | M5 and FA2B | GP2/GP * 100 |
| total | GP3 | A2[6]BG1 | GP3/GP * 100 |
| plasma | GP4 | FA2[6]G1 | GP4/GP * 100 |
| N-glycan | GP5 | FA2[3]G1 | GP5/GP * 100 |
| traits | GP6 | FA2[6]BG1 | GP6/GP * 100 |
| | GP7 | M6 | GP7/GP * 100 |
| | GP8 | A2G2 | GP8/GP * 100 |
| | GP9 | A2BG2 | GP9/GP * 100 |
| | GP10 | FA2G2 | GP10/GP * 100 |
| | GP11 | FA2BG2 | GP11/GP * 100 |
| | GP12 | A2[3]BG1S1 | GP12/GP * 100 |
| | GP13 | FA2[3]G1S1 | GP13/GP * 100 |
| | GP14 | A2G2S1 | GP14/GP * 100 |
| | GP15 | A2BG2S1 | GP15/GP * 100 |
| | GP16 | FA2G2S1 | GP16/GP * 100 |
| | GP17 | FA2BG2S1 | GP17/GP * 100 |
| | GP18 | A2G2S2 | GP18/GP * 100 |
| | GP19 | M9 | GP19/GP * 100 |
| | GP20 | A2G2S2 | GP20/GP * 100 |
| | GP21 | A2BG2S2 | GP21/GP * 100 |
| | GP22 | FA2G2S2 | GP22/GP * 100 |
| | GP23 | FA2BG2S2 | GP23/GP * 100 |
| | GP24 | A3G3S2 | GP24/GP * 100 |
| | GP25 | A3BG3S2 | GP25/GP * 100 |
| | GP26 | A3G3S2 | GP26/GP * 100 |
| | GP27 | A3G3S3 | GP27/GP * 100 |
| | GP28 | A3G3S3 | GP28/GP * 100 |
| | GP29 | FA3G3S3 | GP29/GP * 100 |
| | GP30 | A3G3S3 | GP30/GP * 100 |
| | GP31 | FA3G3S3 | GP31/GP * 100 |
| | GP32 | A3F1G3S3 | GP32/GP * 100 |
| | GP33 | A4G4S3 | GP33/GP * 100 |
| | GP34 | A4G4S3 | GP34/GP * 100 |
| | GP35 | A4F1G3S3 | GP35/GP * 100 |
| | GP36 | A4G4S4 | GP36/GP * 100 |
| | GP37 | A4G4S4 | GP37/GP * 100 |
| | GP38 | A4G4S4 | GP38/GP * 100 |
| | GP39 | A4F1G4S4 | GP39/GP * 100 |
| | | % of Specified Structure | |
| | | in Total Plasma | |
| | | Glycans/Glycome | |
| Derived | S0 | neutral glycan structures | (GP1 + GP2 + GP3 + |
| total | | | GP4 + GP5 + GP6 + |
| plasma | | | GP7 + GP8 + GP9 + |
| N-glycan | | | GP10 + GP11)/GP *100 |
| traits | S1 | monosialylated | (GP12 + GP13 + GP14 + |
| | | structures | GP15 + GP16 + |
| | | | GP17)/GP * 100 |
| | S2 | disialylated structures | (GP18 + GP20 + GP21 + |
| | | | GP22 + GP23 + GP24 + |
| | | | GP25 + GP26)/GP * 100 |
| | S3 | trisialylated structures | (GP27 + GP28 + GP29 + |
| | | | GP30 + GP31 + GP32 + |
| | | | GP33 + GP34 + |
| | | | GP35)/GP * 100 |
| | S4 | tetrasialylated structures | (GP36 + GP37 + GP38 + |
| | | | GP39)/GP * 100 |
| | G0 | agalactosylated | (GP1 + GP2)/GP * 100 |
| | | structures | |
| | G1 | monogalactosylated | (GP3 + GP4 + GP5 + |
| | | structures | GP6 + GP12 + |
| | | | GP13)/GP * 100 |
| | G2 | digalactosylated | (GP8 + GP9 + GP10 + |
| | | structures | GP11 + GP14 + GP15 + |
| | | | GP16 + GP17 + GP18 + |
| | | | GP20 + GP21 + GP22 + |
| | | | GP23)/GP * 100 |
| | G3 | trigalactosylated | (GP24 + GP25 + GP26 + |
| | | structures | GP27 + GP28 + GP29 + |
| | | | GP30 + GP31 + GP32 + |
| | | | GP35)/GP * 100 |
| | G4 | tetragalactosylated | (GP33 + GP34 + GP36 + |
| | | structures | GP37 + GP38 + |
| | | | GP39)/GP * 100 |
| | LB | low branched | (GP1 + GP2 + GP3 + |
| | | (monoantennary and | GP4 + GP5 + GP6 + |
| | | diantennary) structures | GP8 + GP9 + GP10 + |
| | | | GP11 + GP12 + GP13 + |
| | | | GP14 + GP15 + GP16 + |
| | | | GP17 + GP18 + GP20 + |
| | | | GP21 + GP22 + GP23)/ |
| | | | GP * 100 |
| | HB | high branched | (GP24 + GP25 + GP26 + |
| | | (triantennary and | GP27 + GP28 + GP29 + |
| | | tetraantennary) | GP30 + GP31 + GP32 + |
| | | structures | GP33 + GP34 + GP35 + |
| | | | GP36 + GP37 + GP38 + |
| | | | GP39)/GP * 100 |
| | B | structures with bisecting | (GP2 + GP3 + GP6 + |
| | | GlcNAc | GP9 + GP11 + GP12 + |
| | | | GP15 + GP17 + GP21 + |
| | | | GP23 + GP25)/GP * 100 |
| | FUC-A | antennary fucosylated | (GP32 + GP35 + |
| | | structures | GP39)/GP * 100 |
| | FUC-C | core fucosylated | (GP1 + GP2 + GP4 + |
| | | structures | GP5 + GP6 + GP10 + |
| | | | GP11 + GP13 + GP16 + |
| | | | GP17 + GP22 + GP23 + |
| | | | GP29 + GP31)/GP * 100 |

The Key for Tables 2 and 3 is as follows:

GP=SUM (GP1:GP24) for Table 2; GP=SUM (GP1:GP39) for Table 3;
F—fucose, A—antennary N-acetylglucosamine (GlcNAc), M—mannose, G—galactose, B—bisecting GlcNAc, S—N-acetylneuraminic acid (sialic acid).

Number after the letter represents number of sugars in the glycan structure.

E. Statistical Analyses.

Data were analyzed using Prism 7.0 (GraphPad Software, La Jolla, Calif., USA). Nonparametric two-tailed Mann-Whitney U-test was used for estimating significance of glycan abundance difference between pairs of patient groups and False Discovery Rate (FDR) for correction for multiple testing was done using Storey procedure⁶² p<0.05 and FDR<20% results were considered significant. Associations between glycan abundance and other patient measurements and characteristics was done using Spearman's rank correlation with p<0.05 used as a significance threshold.

### Results of the Examples

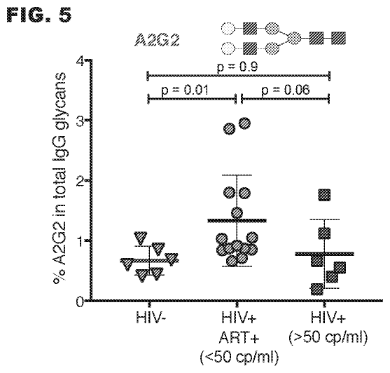

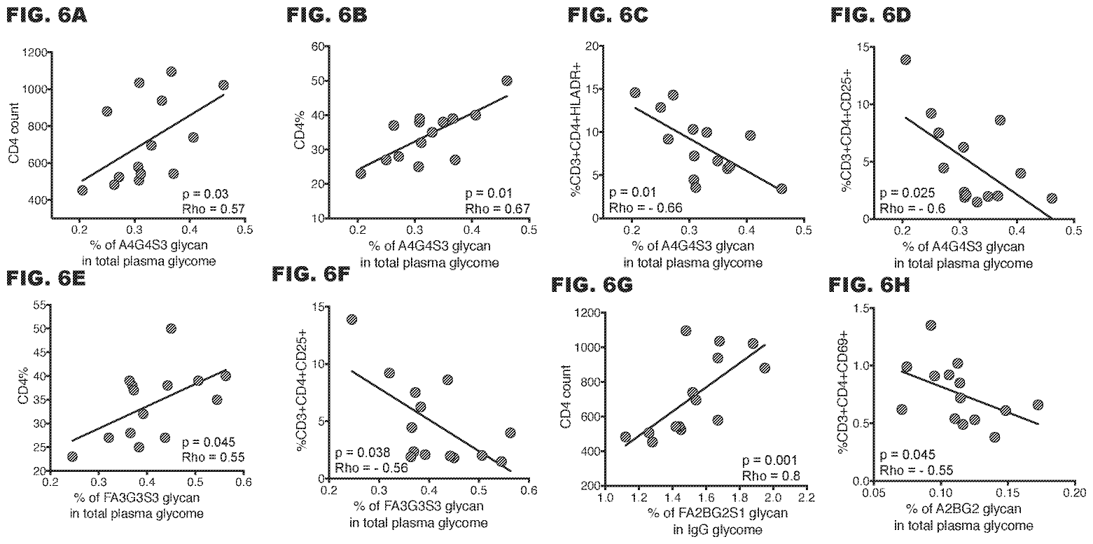

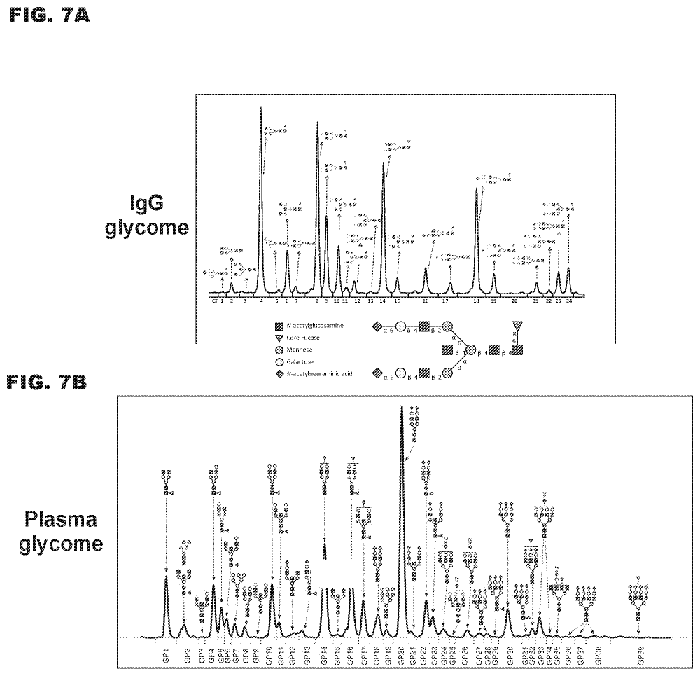

HIV infection is associated with persistent alterations in the IgG glycome. The glycomes of isolated IgG from HIV-negative controls, HIV+ ART-suppressed individuals (viral load<20 copies/ml), and HIV+ unsuppressed individuals (viral load=>50 copies/ml) (See Table 1) were profiled using UPLC. IgG N-glycans were separated into 24 chromatographic peaks [30] (FIG. 7A). Each peak represents a glycan trait. Certain IgG glycan traits were combined into categories, glycan structures that contain galactose (G0=agalactosylated, G1=mono-galactosylated, G2=di-galactosylated) or that contain sialic acid (S=sialylated), fucose (F=fucosylated), or a bisecting GlcNAc (B=bisected) (Table 2).

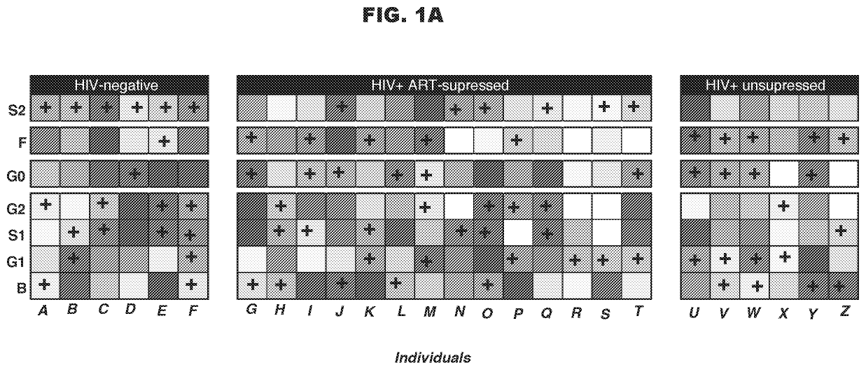

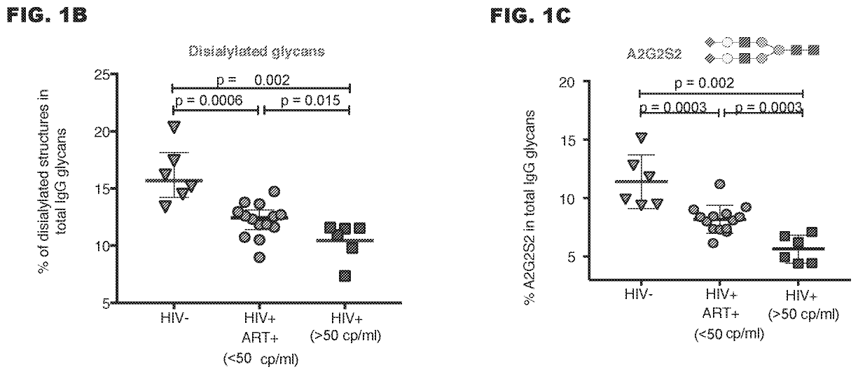

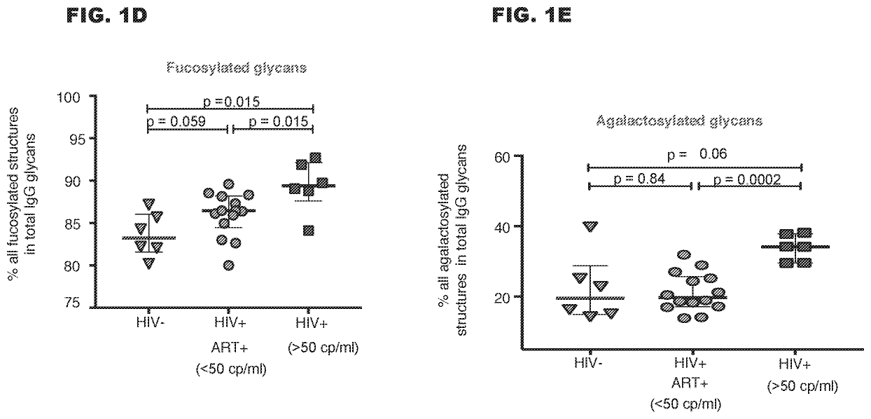

Levels of di-sialylated glycans in total IgG glycome were significantly reduced in HIV+unsuppressed individuals (p=0.002, median=11.2%, IQR=1.4), and HIV+ART-suppressed individuals (p=0.0006, median=12.4%, IQR=1.2), when compared to HIV-negative controls (median=15.7%, IQR=2.4) (FIGS. 1A and 1B). Out of IgG di-sialylated glycan structures, the level of A2G2S2 glycan trait (glycan structure with two sialic acids, two galactoses, and no core fucose or bisecting GlcNAc) was significantly reduced in the IgG glycomes of HIV+ unsuppressed individuals (p=0.002, median=5.6%, IQR=2), and HIV+ ART-suppressed individuals (p=0.0003, median=8.2%, IQR=1.2), when compared to HIV-negative controls (median=10.8%, IQR=3) (FIGS. 1A and 1C).

Levels of IgG fucosylated glycans were significantly induced in HIV+ unsuppressed individuals (p=0.015, median=89.4%, IQR=2.5), and trending in HIV+ ART-suppressed individuals (p=0.059, median=86.4%, IQR=2.7), when compared to HIV-negative controls (median=83.2%, IQR=3.2) (FIGS. 1A and 1D).

Finally, as reported before^{58, 59}, levels of agalactosylated glycans were induced in HIV+ unsuppressed individuals (median=34.1%, IQR=6.1), when compared to HIV+ ART-suppressed individuals (p=0.0002, median=19.7%, IQR=7.5) and trending when compared to HIV-negative controls (p=0.06, median=19.5%, IQR=9.2) (FIGS. 1A and 1E). These results support new IgG glycomic alterations that associate with HIV infection and persistent despite suppressive ART. These new associations with HIV infection are, in one embodiment, decreased levels of IgG sialylated glycans, which is associated with a pro-inflammatory activity¹³⁻¹⁵, and in another embodiment, increased levels of IgG fucosylated glycans, which is associated with lower ADCC and higher inflammation, regardless of the ART-suppression status.

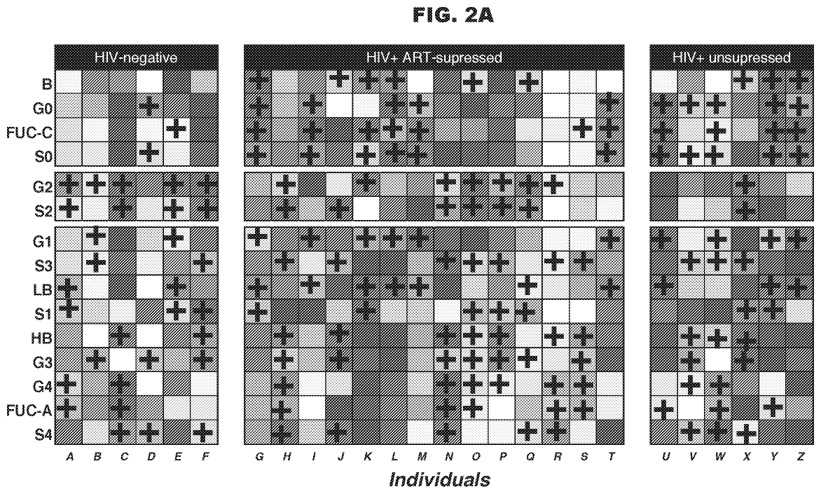

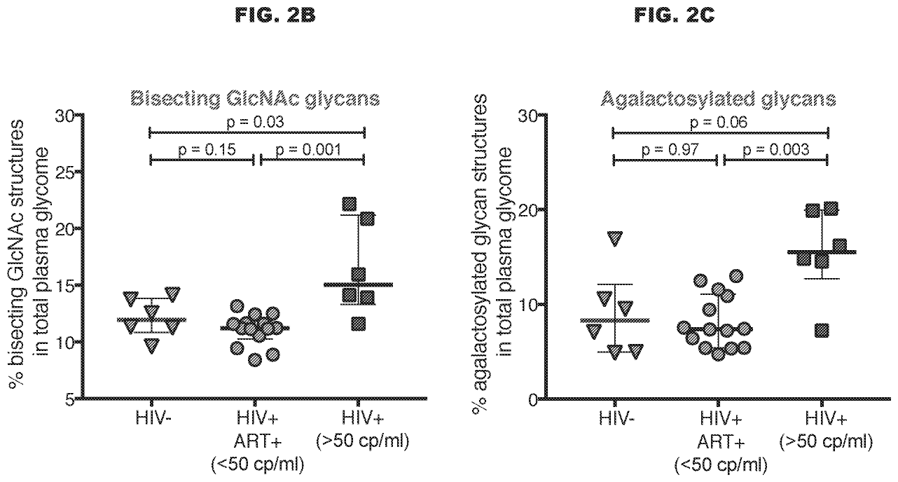

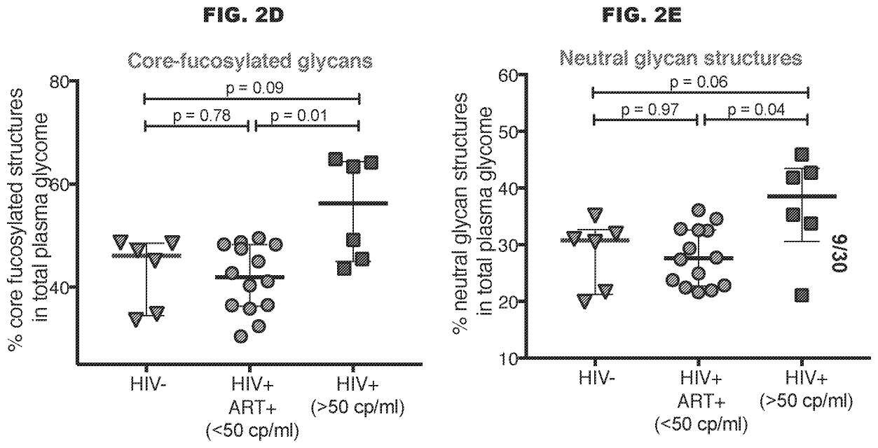

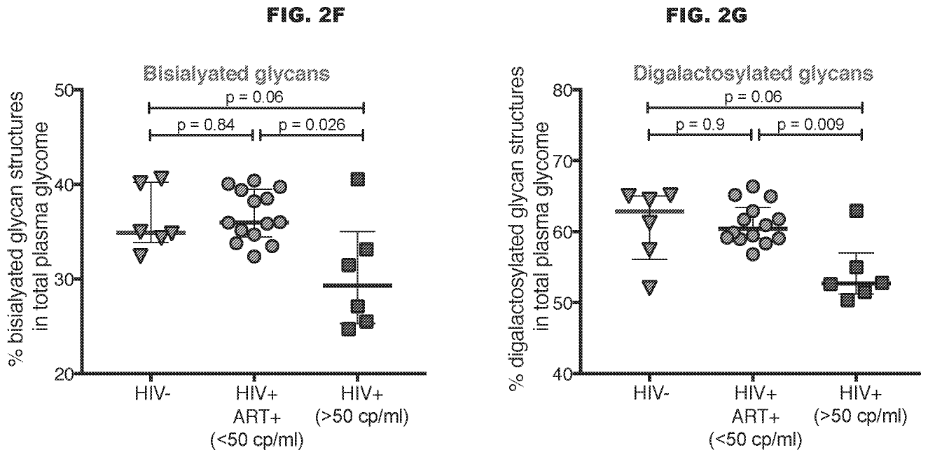

Plasma glycomic alterations are associated with HIV infection. The glycomes of plasma (FIGS. 2A-2G) from the same individuals as above were profiled using UPLC. Total plasma N-glycans were chromatographically separated into 39 peaks (FIG. 7B). Each peak represents a glycan trait. Additionally, structurally similar glycan species have been combined into derived traits to assess changes in the levels of galactosylation, sialylation, fucosylation, bisecting GlcNAc and branching (See, FIG. 2A and Table 3).

Levels of several plasma glycan structures were modulated in HIV+ unsuppressed individuals when compared to both HIV+ ART-suppressed and HIV-negative controls, suggesting reversible changes when measured at the total plasma level. Levels of plasma bisecting GlcNAc, agalactosylated, core-fucosylated, neutral (no-sialic-acid) glycan structures were induced in HIV+ unsuppressed individuals when compared to both HIV+ ART-suppressed and HIV-negative controls (p<0.05) (See FIGS. 2A-2E). Levels of plasma di-sialylated and di-galactosylated glycan structures were reduced in HIV+ unsuppressed individuals when compared to both HIV+ ART-suppressed and HIV-negative controls (p<0.05) (See FIGS. 2A, 2E-2G).

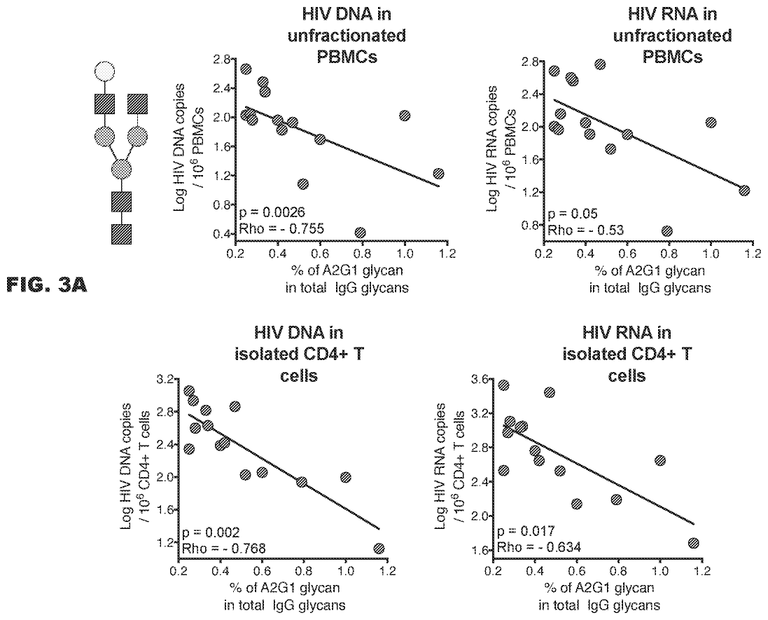

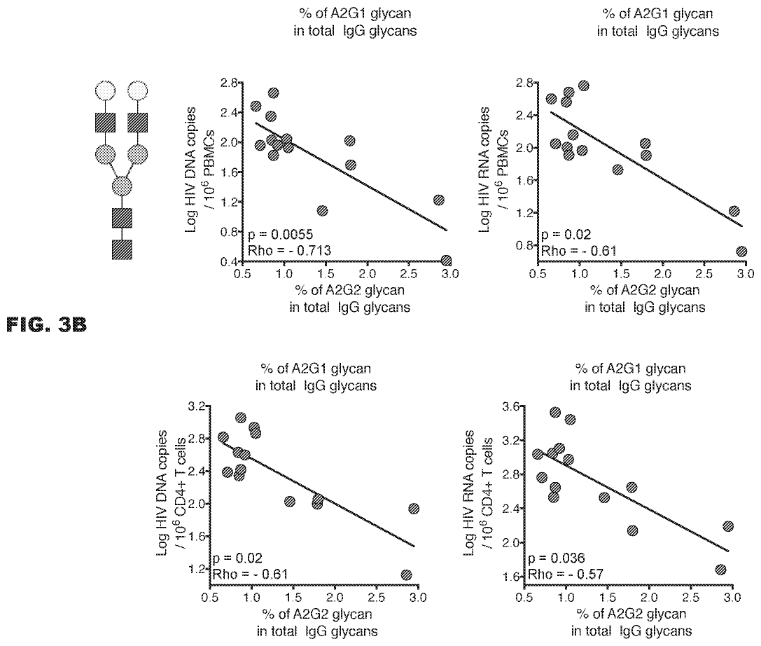

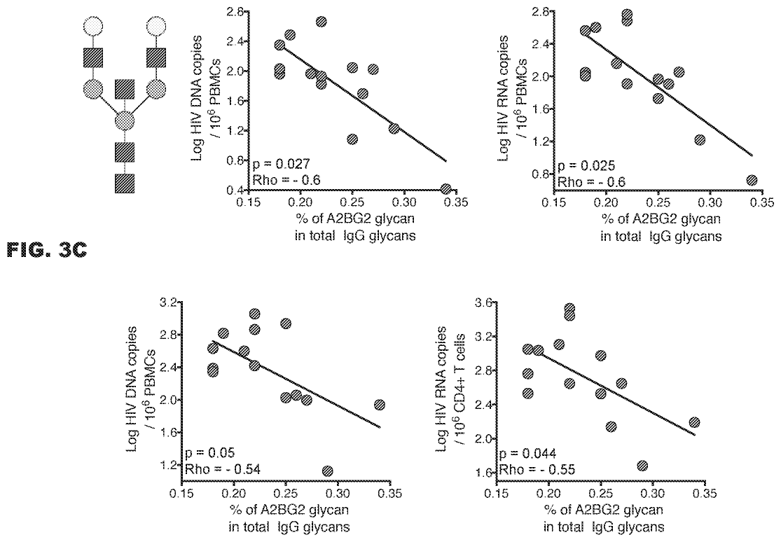

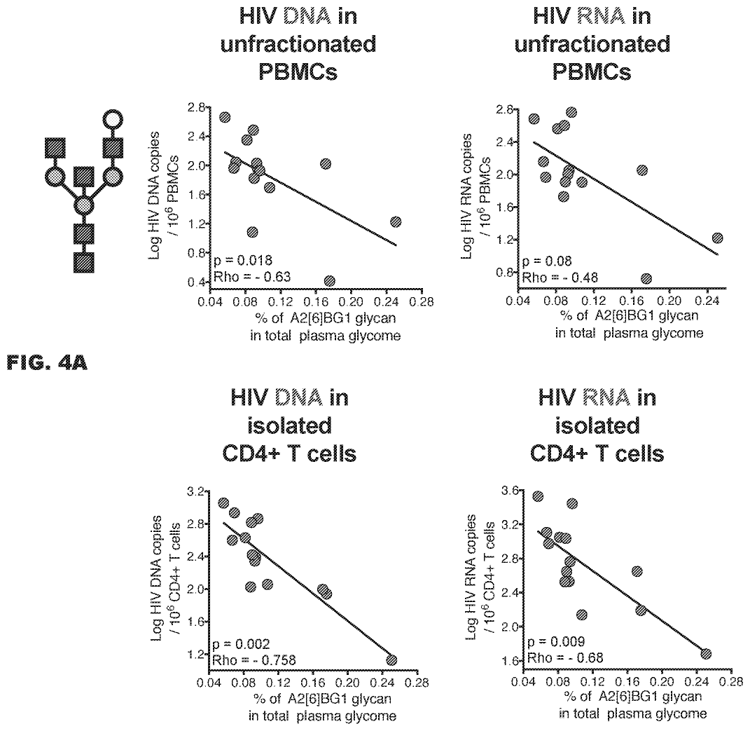

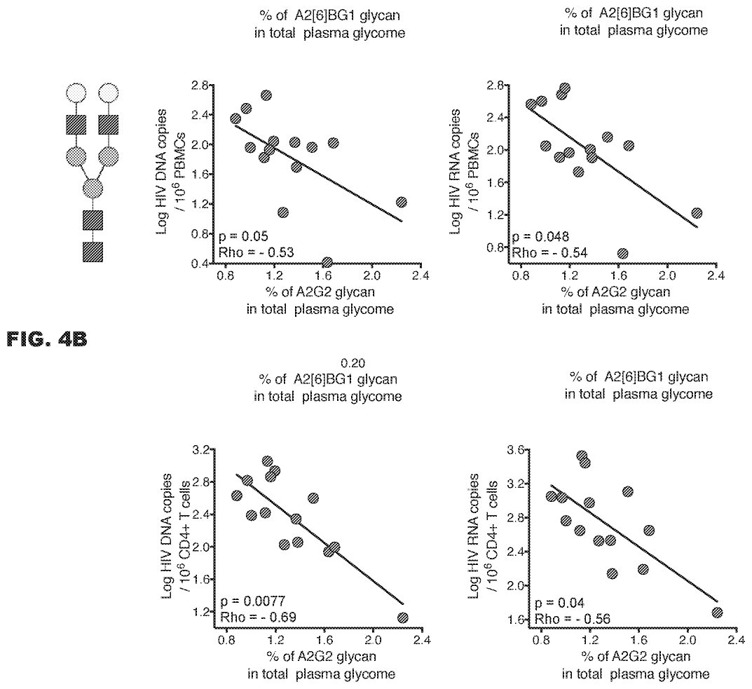

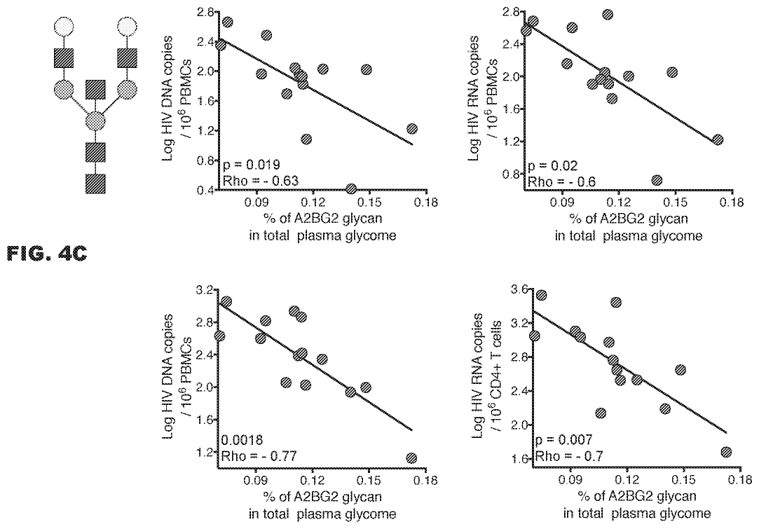

Levels of glycomic traits in isolated IgG and total plasma glycomes correlate with nucleic acid measures of HIV persistence during ART. To test the hypothesis that glycomic alterations in plasma or IgG can affect HIV reservoir size during suppressive ART, we examined relationships between nucleic acid measures of the latent reservoir and both the glycomes of isolated IgG and total plasma. Levels of cell-associated HIV total DNA and HIV RNA were measured in unfractionated PBMCs and negatively-selected peripheral blood CD4+ T cells from HIV+ ART-suppressed individuals within the above-mentioned cohort. Levels of total HIV DNA and cell-associated HIV RNA in both unfractionated PBMC and isolated CD4+ were negatively correlated with levels of three glycan traits in isolated IgG (FIGS. 3A-3C) and three glycan traits in total plasma (FIGS. 4A-4C). All these glycan structures are similar in that they are mono- or di-galactosylated, with no sialic acid or core fucose, and two of these three glycan structures are the same in isolated IgG and total plasma (A2G2 and A2BG2).

Levels of A2G1 (monogalactosylated glycan structure with no sialic acid or core fucose) in total IgG glycome inversely correlated with levels of total HIV DNA in unfractionated PBMC (p=0.0026, rho=−0.755) and isolated CD4+ T cells (p=0.002, rho=−0.768), and inversely correlated with levels of cell-associated HIV RNA in unfractionated PBMC (p=0.05, rho=−0.53) and isolated CD4+ T cells (p=0.017, rho=−0.634) (as shown in FIG. 3A). Levels of A2G2 (di-galactosylated glycan structure with no sialic acid or core fucose) in total IgG glycome inversely correlated with levels of total HIV DNA in unfractionated PBMC (p=0.0055, rho=−0.713) and isolated CD4+ T cells (p=0.02, rho=−0.61), and inversely correlated with levels of cell-associated HIV RNA in unfractionated PBMC (p=0.02, rho=−0.61) and isolated CD4+ T cells (p=0.036, rho=−0.57) (FIG. 3B).

Levels of A2BG2 (di-galactosylated structure with bisecting GlcNAc, with no sialic acid or core fucose) in total IgG glycome inversely correlated with levels of total HIV DNA in unfractionated PBMC (p=0.027, rho=−0.6) and isolated CD4+ T cells (p=0.05, rho=−0.54), and inversely correlated with levels of cell-associated HIV RNA in unfractionated PBMC (p=0.025, rho=−0.6) and isolated CD4+ T cells (p=0.044, rho=−0.55) (FIG. 3C).

Levels of A2[6]BG1 (monogalactosylated structure with bisecting GlcNAc, with no sialic acid or core fucose) in total plasma glycome inversely correlated with levels of total HIV DNA in unfractionated PBMC (p=0.018, rho=−0.63) and isolated CD4+ T cells (p=0.002, rho=−0.758), and inversely correlated with levels of cell-associated HIV RNA in isolated CD4+ T cells (p=0.009, rho=−0.68) (FIG. 4A). Levels of A2G2 (di-galatosylated glycan structure, with no sialic acid or core fucose) in total plasma glycome inversely correlated with levels of total HIV DNA in unfractionated PBMC (p=0.05, rho=−0.53) and isolated CD4+ T cells (p=0.0077, rho=−0.69), and inversely correlated with levels of cell-associated HIV RNA in unfractionated PBMC (p=0.048, rho=−0.54) and isolated CD4+ T cells (p=0.04, rho=−0.56) (FIG. 4B). Levels of A2BG2 (di-galactosylated glycan structure with bisecting GlcNAc, with no sialic acid or core fucose) in total plasma glycome inversely correlated with levels of total HIV DNA in unfractionated PBMC (p=0.019, rho=−0.63) and isolated CD4+ T cells (p=0.0018, rho=−0.77), and inversely correlated with levels of cell-associated HIV RNA in unfractionated PBMC (p=0.02, rho=−0.6) and isolated CD4+ T cells (p=0.007, rho=−0.7) (FIG. 4C).

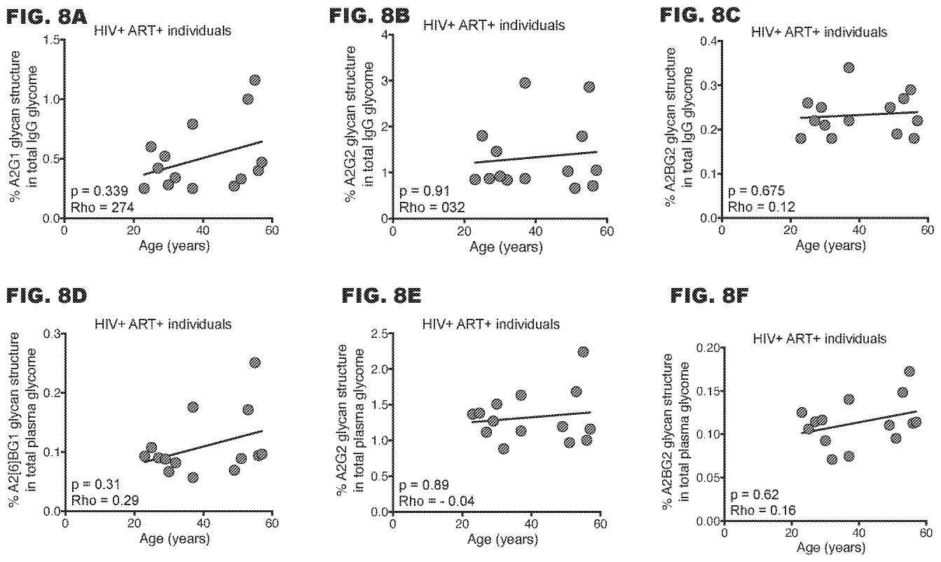

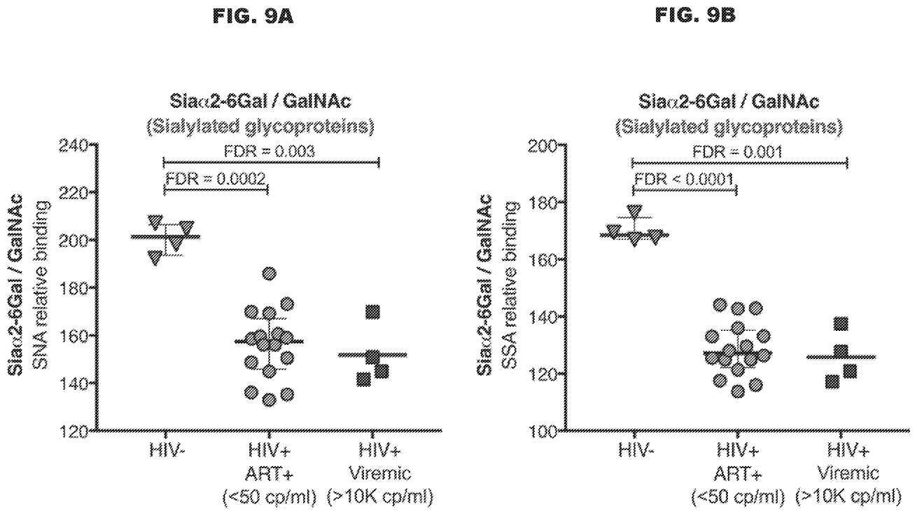

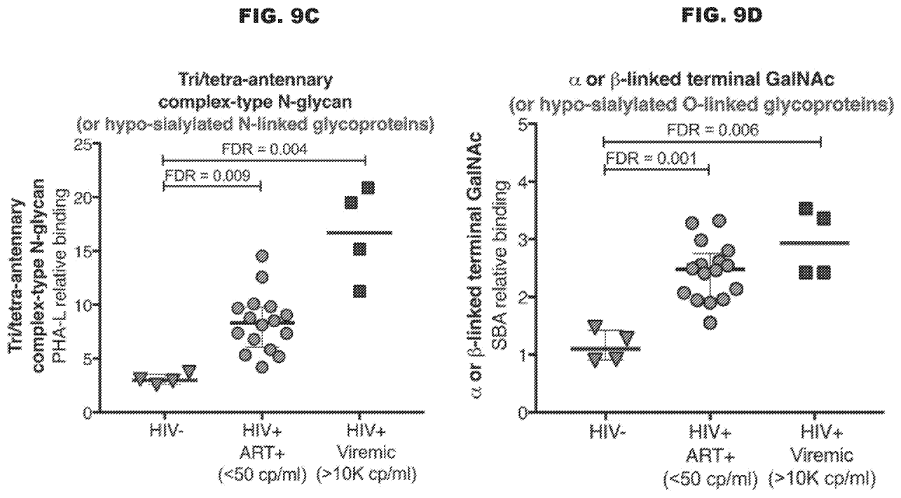

Interestingly, the levels of A2G2 were higher in the IgG glycomes of HIV+ ART-suppressed individuals (median=1%, IQR=0.9) when compared to HIV-negative controls (p=0.01, median=0.6%, IQR=0.3), and trend higher when compared to HIV+ unsuppressed individuals (p=0.06, median=0.6%, IQR=0.6 as shown in FIG. 5). To test if age played a role in the observed relationships, we examined the correlations between the expression of these glycan traits and age. No correlation was detected between age and levels of A2G1, A2G2, A2BG2 in isolated IgG glycome or levels of A2[6]BG1, A2G2, and A2BG2 in total plasma glycome (See FIGS. 8A-8F). In addition, all significant glycan associations with HIV DNA/RNA loads were tested using linear regression with both glycan abundance and age as factors, and none of the results lost significance indicating independence of glycan association from age.

Levels of circulating anti-inflammatory glycans associate with higher levels of CD4 count and lower levels of T cell activation. Multiparametric flow cytometry was used to assess the percentage of three activation markers that have been associated with HIV latency, i.e., HLA-DR, CD69, and CD25 on CD4+ T cells. The percentage of sialylated glycans (FA2BG2S1 in IgG; FA3G3S3 and A4G4S3 in total plasma) and galactosylated glycans (A2BG2 in total plasma), glycans associated with anti-inflammatory activities¹³⁻¹⁶, exhibited significant positive correlations with CD4 count, CD4%, and inverse correlations with the expression of several CD4+ T cell activation markers in HIV+ ART-suppressed individuals (See FIGS. 6A-6H).

Levels of A4G4S3 in total plasma correlated with CD4 count (p=0.03, rho=0.57), CD4% (p=0.01, rho=0.67), and inversely correlated with the percentage of CD4+ T cells expressing the HLA-DR late activation marker (p=0.01, rho=−0.66) or the intermediate or late CD25 activation marker (p=0.025, rho=−0.6) (FIGS. 6A-6D). Levels of FA3G3S3 in total plasma positively correlated with CD4% (p=0.045, rho=0.55), and inversely correlated with the percentage of CD4+ T cells expressing the CD25 activation marker (p=0.038, rho=−0.56) (FIGS. 6E-F). Levels of FA2BG2S in IgG glycome positively correlated with CD4 count (p=0.001, rho=0.8) (FIG. 6G). Levels of A2BG2 in total plasma inversely correlated with the percentage of CD4+ T cells expressing the CD69 early activation marker (p=0.045, rho=−0.55) (FIG. 6H).

Each and every patent, patent application and any document listed herein, and the sequence of any publicly available nucleic acid and/or peptide sequence cited throughout the disclosure, is/are expressly incorporated herein by reference in its entirety to provide teachings extant in the field. Embodiments and variations of this invention other than those specifically disclosed above may be devised by others skilled in the art without departing from the true spirit and scope of the invention. The appended claims include such embodiments and equivalent variations.

### REFERENCES

1. May, M. T et al, Impact on Life Expectancy of Hiv-1 Positive Individuals of Cd4+ Cell Count and Viral Load Response to Antiretroviral Therapy. Aids 28, 1193-1202, Doi:10.1097/Qad.0000000000000243 (2014).

2. Kuller, L. H., et. al., Inflammatory and Coagulation Biomarkers and Mortality in Patients with HIV Infection. Plos Medicine 5, E203, Doi:0.1371/Journal.Pmed .0050203 (2008).

3. Piketty, C., et al., Long-Term Clinical Outcome of Human Immunodeficiency Virus-Infected Patients with Discordant Immunologic and Virologic Responses to a Protease Inhibitor-Containing Regimen. The Journal of Infectious Diseases 183, 1328-1335, Doi:10.1086/319861 (2001).

4. Engsig, F. N., et al., Long-Term Mortality in HIV Patients Virally Suppressed for More Than Three Years with Incomplete CD4 Recovery: A Cohort Study. Bmc Infectious Diseases 10, 318, Doi:10.1186/1471-2334-10-318 (2010).

5. Barrera, C., et al., Differential Glycosylation of MHC Class II Molecules on Gastric Epithelial Cells: Implications in Local Immune Responses. Human Immunology 63, 384-393 (2002).

6. De Freitas Junior, J. C., et al., Inhibition Of N-Linked Glycosylation by Tunicamycin Induces E-Cadherin-Mediated Cell-Cell Adhesion and Inhibits Cell Proliferation in Undifferentiated Human Colon Cancer Cells. Cancer Chemotherapy and Pharmacology 68, 227-238, Doi:10.1007/S00280-010-1477-8 (2011).

7. Dwek, R. A., et al., Targeting Glycosylation as a Therapeutic Approach. Nature Reviews. Drug Discovery 1, 65-75, Doi:10.1038/Nrd708 (2002).

8. Azuma, Y., et al., Immobilized Alpha2,6-Linked Sialic Acid Suppresses Caspase-3 Activation During Anti-IgM Antibody-Induced Apoptosis in Ramos Cells. Biochimica Et Biophysica Acta 1770, 279-285, Doi:10.1016/J.Bbagen.2006.10.010 (2007).

9. Lichtenstein, R. G. & Rabinovich, G. A. Glycobiology Of Cell Death: When Glycans And Lectins Govern Cell Fate. Cell Death Differ 20, 976-986, Doi:10.1038/Cdd.2013.50 (2013).

10. Suzuki, 0., et al., Caspase-Dependent Drug-Induced Apoptosis Is Regulated by Cell Surface Sialylation in Human B-Cell Lymphoma. Oncol Lett 10, 687-690, Doi:10.3892/01.2015.3320 (2015).

11. Zhuo, Y. & Bellis, S. L. Emerging Role of Alpha2,6-Sialic Acid as a Negative Regulator of Galectin Binding and Function. The Journal of Biological Chemistry 286, 5935-5941, Doi:10.1074/Jbc.R110.191429 (2011).

12. Chan, A. C. & Carter, P. J. Therapeutic Antibodies for Autoimmunity and Inflammation. Nature Reviews. Immunology 10, 301-316, Doi:10.1038/Nri2761 (2010).

13. Anthony, R. M., et al., Recapitulation of IVIG Anti-Inflammatory Activity with a Recombinant IgG Fc. Science 320, 373-376, Doi:10.1126/Science.1154315 (2008).

14. Kaneko, Y., et al., Anti-Inflammatory Activity of Immunoglobulin G Resulting from Fc Sialylation. Science 313, 670-673, Doi:10.1126/Science.1129594 (2006).

15. Washburn, N., et al., Controlled Tetra-Fc Sialylation of IVIG Results in a Drug Candidate with Consistent Enhanced Anti-Inflammatory Activity. Proceedings of The National Academy of Sciences of The United States of America 112, E1297-1306, Doi:10.1073/Pnas.1422481112 (2015).

16. Karsten, C. M., et al., Anti-Inflammatory Activity of IgG1 Mediated by Fc Galactosylation and Association of Fc gamma RIIB And Dectin-1. Nature Medicine 18, 1401-1406, Doi:10.1038/Nm.2862 (2012).

17. Appay, V., et al. Old Age and Anti-Cytomegalovirus Immunity Are Associated with Altered T-Cell Reconstitution in Hiv-l-Infected Patients. Aids 25, 1813-1822, Doi:10.1097/Qad.0b013e32834640e6 (2011).

18. Kelley, C. F., et al., Incomplete Peripheral Cd4+ Cell Count Restoration in HIV-Infected Patients Receiving Long-Term Antiretroviral Treatment. Clinical Infectious Diseases: An Official Publication of The Infectious Diseases Society of America 48, 787-794, Doi:10.1086/597093 (2009).

19. Hunt, P. W., et al., T Cell Activation Is Associated with Lower CD4+ T Cell Gains in Human Immunodeficiency Virus-Infected Patients with Sustained Viral Suppression During Antiretroviral Therapy. The Journal of Infectious Diseases 187, 1534-1543, Doi:10.1086/374786 (2003).

20. Marziali, M., et al., T-Cell Homeostasis Alteration In Hiv-1 Infected Subjects With Low Cd4 T-Cell Count Despite Undetectable Virus Load During Haart. Aids 20, 2033-2041, Doi:10.1097/01. AIDS.0000247588.69438.Fd (2006).

21. Benito, J. M., et al., Cd4+ T Cell Recovery Beyond the First Year of Complete Suppression of Viral Replication During Highly Active Antiretroviral Therapy Is Not Influenced by CD8+ T Cell Activation. The Journal of Infectious Diseases 192, 2142-2146, Doi:10.1086/498168 (2005).

22. Poor CD4 Cell Recovery and Persistent Inflammation Despite Viral Suppression. Guide. Use Antiretrovir. Agents Hiv-1-Infected Adults Adolesc. Guidel. Use Antiretrovir. Agents Hiv-1-Infected Adults Adolesc.

23. Williams, G. J. & Thorson, J. S. Natural Product Glycosyltransferases: Properties and Applications. Advances in Enzymology and Related Areas of Molecular Biology 76, 55-119 (2009).

24. An, H. J. & Lebrilla, C. B. A Glycomics Approach to The Discovery of Potential Cancer Biomarkers. Methods in Molecular Biology 600, 199-213, Doi:10.1007/978-1-60761-454-8_14 (2010).

25. Moremen, K. W., et al., Vertebrate Protein Glycosylation: Diversity, Synthesis, and Function. Nature Reviews. Molecular Cell Biology 13, 448-462, Doi:10.1038/Nrm3383 (2012).

26. Pucic, M., et al., High Throughput Isolation and Glycosylation Analysis of IgG-Variability and Heritability of the IgG Glycome in Three Isolated Human Populations. Molecular & Cellular Proteomics : Mcp 10, M111 010090, Doi:10.1074/Mcp.M111. 010090 (2011).

27. Goede, V., et al., Obinutuzumab Plus Chlorambucil in Patients With CLL and Coexisting Conditions. The New England Journal of Medicine 370, 1101-1110, Doi:10.1056/Nejmoa1313984 (2014).

28. Junttila, T. T., et al., Superior In Vivo Efficacy of Afucosylated Trastuzumab in the Treatment of Her2-Amplified Breast Cancer. Cancer Research 70, 4481-4489, Doi:10.1158/0008-5472.Can-09-3704 (2010).

29. Scott, A. M., et al., Antibody Therapy of Cancer. Nature Reviews. Cancer 12, 278-287, Doi:10.1038/Nrc3236 (2012).

30. Sondermann, P. & Szymkowski, D. E. Harnessing Fc Receptor Biology in The Design of Therapeutic Antibodies. Current Opinion in Immunology 40, 78-87, Doi:10.1016/J. Coi.2016.03.005 (2016).

31. Haga, C. L., et al., Fc Receptor-Like 5 Inhibits B Cell Activation Via Shp-1 Tyrosine Phosphatase Recruitment. Proceedings of The National Academy of Sciences of The United States of America 104, 9770-9775, Doi:10.1073/Pnas.0703354104 (2007).

32. Massoud, A. H., et al., Dendritic Cell Immunoreceptor: A Novel Receptor for Intravenous Immunoglobulin Mediates Induction of Regulatory T Cells. The Journal of Allergy and Clinical Immunology 133, 853-863 E855, Doi: 10.1016/J.Jaci.2013.09.029 (2014).

33. Seite, J. F., et al., IVIG Modulates BCR Signaling Through CD22 And Promotes Apoptosis in Mature Human B Lymphocytes. Blood 116, 1698-1704, Doi:10.1182/Blood-2009-12-261461 (2010).

34. Varki, A. & Gagneux, P. Multifarious Roles of Sialic Acids in Immunity. Annals of The New York Academy of Sciences 1253, 16-36, Doi:10.1111/11749-6632.2012.06517.X (2012).

35. Baker, J. V., et al., Immunologic Predictors of Coronary Artery Calcium Progression in a Contemporary HIV Cohort. Aids 28, 831-840, Doi:10.1097/Qad.0000000000000145 (2014).

36. Barbour, J. D., et al., Reduced Cd14 Expression on Classical Monocytes and Vascular Endothelial Adhesion Markers Independently Associate with Carotid Artery Intima Media Thickness in Chronically HIV-1 Infected Adults on Virologically Suppressive Anti-Retroviral Therapy. Atherosclerosis 232, 52-58, Doi:10.1016/ J. Atherosclerosis.2013.10.021 (2014).

37. D′abramo, A., et al., Immune Activation, Immunosenescence, and Osteoprotegerin as Markers of Endothelial Dysfunction in Subclinical HIV-Associated Atherosclerosis. Mediators Inflamm 2014, 192594, Doi:10.1155/2014/192594 (2014).

38. Gresele, P., et al., Endothelial and Platelet Function Alterations In HIV-Infected Patients. Thromb Res 129, 301-308, Doi:10.1016/J.Thromres.2011.11.022 (2012).

39. Manion, M., et al., Country of Residence Is Associated with Distinct Inflammatory Biomarker Signatures in HIV-Infected Patients. J Virus Erad 3, 24-33 (2017).

40. Mckibben, R. A., et al., Elevated Levels of Monocyte Activation Markers Are Associated with Subclinical Atherosclerosis in Men with And Those without HIV Infection. J Infect Dis 211, 1219-1228, Doi:10.1093/Infdis/Jiu594 (2015). 41. Neuhaus, J., et al., Markers of Inflammation, Coagulation, and Renal Function Are Elevated in Adults with HIV Infection. J Infect Dis 201, 1788-1795, Doi:10.1086/652749 (2010).

42. Spence, S., et al., Targeting Siglecs With a Sialic Acid-Decorated Nanoparticle Abrogates Inflammation. Sci Transl Med 7, 303ra140, Doi:10.1126/Scitranslmed. Aab3459 (2015).

43. Kim, Y. H., et al., Development of Sialic Acid-Coated Nanoparticles for Targeting Cancer and Efficient Evasion of The Immune System. Theranostics 7, 962-973, Doi:10.7150/Thno.19061 (2017).

44. Belaunzaran-Zamudio, P. F., et al., Immunologic Effects of Maraviroc in HIV-Infected Patients with Severe CD4 Lymphopenia Starting Antiretroviral Therapy: A Sub-Study of The CADIRIS Trial. Pathog Immun 2, 151-177, Doi:10.20411/ Pai.V2i2.181 (2017).

45. Patro, S. C., et al., J. Antiretroviral Therapy in HIV-1-Infected Individuals with CD4 Count Below 100 Cells/Mm³ Results in Differential Recovery of Monocyte Activation. J Leukoc Biol 100, 223-231, Doi:10.1189/J1b.5ab0915-406r (2016).

46. Sierra-Madero, et al., A Randomized, Double-Blind, Placebo-Controlled Clinical Trial of a Chemokine Receptor 5 (Ccr5) Antagonist to Decrease the Occurrence of Immune Reconstitution Inflammatory Syndrome in HIV-Infection: The CADIRIS Study. Lancet HIV 1, E60-E67, Doi:10.1016/S2352-3018(14)70027-X (2014).

47. Sierra-Madero, J. G., et al., Effect of The Ccr5 Antagonist Maraviroc On the Occurrence of Immune Reconstitution Inflammatory Syndrome in HIV (CADIRIS): A Double-Blind, Randomized, Placebo-Controlled Trial. Lancet HIV 1, E60-67, Doi:10.1016/52352-3018(14)70027-X (2014).

48. Angeloni, S., et al., Glycoprofiling With Micro-Arrays of Glycoconjugates and Lectins. Glycobiology 15, 31-41, Doi:10.1093/Glycob/Cwh143 (2005).

49. Chen, P., et al., Identification Of N-Glycan of Alpha-Fetoprotein by Lectin Affinity Microarray. J Cancer Res Clin Oncol 134, 851-860, Doi:10.1007/500432-008-0357-7 (2008).

50. Chen, S., et al., Analysis of Cell Surface Carbohydrate Expression Patterns in Normal and Tumorigenic Human Breast Cell Lines Using Lectin Arrays. Anal Chem 79, 5698-5702, Doi:10.1021/Ac070423k (2007).

51. Fromell, K., et al., Nanoparticle Decorated Surfaces with Potential Use in Glycosylation Analysis. Colloids Surf B Biointerfaces 46, 84-91, Doi:10.1016/J.Colsurfb.2005.06.017 (2005).

52. Kuno, A., et al., Evanescent-Field Fluorescence-Assisted Lectin Microarray: A New Strategy for Glycan Profiling. Nat Methods 2, 851-856, Doi:10.1038/Nmeth803 (2005).

53. Matsuda, A., et al., Development of An All-In-One Technology for Glycan Profiling Targeting Formalin-Embedded Tissue Sections. Biochem Biophys Res Commun 370, 259-263, Doi:10.1016/J.Bbrc.2008.03.090 (2008).

54. Pilobello, K. T., et al., Development of A Lectin Microarray for The Rapid Analysis of Protein Glycopatterns. Chembiochem 6, 985-989, Doi:10.1002/Cbic.200400403 (2005).

55. Zheng, T., et al., Lectin Arrays for Profiling Cell Surface Carbohydrate Expression. J Am Chem Soc 127, 9982-9983, Doi:10.1021/Ja0505550 (2005).

56. Akmacic, I. T., et al., High-Throughput Glycomics: Optimization of Sample Preparation. Biochemistry (Mosc) 80, 934-942, Doi:10.1134/S0006297915070123 (2015).

57. Trbojevic-Akmacic, I., et al., Comparative Analysis and Validation of Different Steps in Glycomics Studies. Methods Enzymol 586, 37-55, Doi:10.1016/Bs.Mie.2016.09.027 (2017).

58. Ackerman, M. E., et al., (2013) Natural variation in Fc glycosylation of HIV-specific antibodies impacts antiviral activity. The Journal of clinical investigation 123, 2183-92

59. Moore, J. S., et al. (2005) Increased levels of galactose-deficient IgG in sera of HIV-1-infected individuals. Aids 19, 381-9.

60. Zhang, L, et al, 2016 Glycan analysis of therapeutic glycoproteins, MABS, 8(2):205-215

61. Novokmet et al, 2014, Changes in IgG and total plasma protein glycomes in acute systemic inflammation, Sci Rep, 4:4347

62. Storey, JD 2003, The Positive False Discovery Rate: a Bayesian Interpretation and the q-value. The Annals of Stats., 31(6):2013-2035

1. An in vitro method for identifying or monitoring HIV persistence or the development of an HIV-comorbidity in an HIV+ subject, comprising:
generating a glycomic signature characterized by the level of selected single glycan structure or multiple glycan structures within a biological sample obtained from the HIV+ subject or within a component of the sample; and
determining the modification of certain glycan structures ithin the sample compared with that from a control,
wherein selected modification of the glycomic signature is an indication of developing an HIV-comorbidity.

2. A method for treating HIV persistence or the development of an HIV-comorbidity in an HIV+ subject, comprising:
generating a glycomic signature characterized by the level of selected single glycan structure or multiple glycan structures within a biological sample obtained from the HIV+ subject or within a component of the sample;
determining the modification of certain glycan structures within the sample compared with that from a control,
wherein selected modification of the glycomic signature is an indication of developing an HIV-comorbidity;
and treating the subject by modifying or normalizing of a selected glycan or multiple glycans in the subject's glycome.

3. The method according to claim 1, wherein the HIV comorbidity is an age-associated disease, inflammation-associated disease, or immune-activation-associated disease.

4. The method according to claim 1, wherein the subject has received antiretroviral therapy (ART) before or during the occurrence of the disease.

5. The method according to claim 1, wherein the sample comprises:
(a) the subject's total plasma glycome,
(b) the subject's total IgG glycome,
(c) the subject's cell-surface glycome from all cells or from a selected cell type; or
(d) the subject's total exosome-bound glycome

6. The method according to claim 1, wherein the selected modification is hyposialylation, which is indicative of HIV persistence.

7. The method according to claim 1, wherein the selected modification is hypo-galactosylation, which is indicative of development a large reservoir of HIV and thus HIV persistence.

8. The method according to claim 1, wherein the selected modification is an increase in fucosylation.

9. A method for treating an HIV-infected subject comprising modifying or normalizing the level of a selected glycan or multiple glycans in the subject's glycome.

10. The method according to claim 9, further comprising normalizing the level of a selected glycan to that of an uninfected control, an Immune Responder control, or controls negative for an HIV comorbidity.

11. The method according to claim 9, wherein the selected glycan is sialic acid.

12. The method according to claim 9, wherein the selected glycan is galactose.

13. The method according to claim 9, wherein the selected glycan is fucose.

14. The method according to claim 9, wherein the method prevents the early development of inflammation- and inflammation-associated diseases in HIV+ individuals or reduces the size of the HIV reservoir.

15. (canceled)

16. The method according to claim 9, wherein the method decreases the levels of immune activation and dysregulation in HIV+ individuals.

17. The method according to claim 9, further comprising increasing or decreasing the amount of the selected glycan in vivo during, before or subsequent to treatment of the subject with ART.

18. The method according to claim 9, comprising administering a therapeutic agent to said subject to modify the selected glycan.

19. The method according to claim 9, comprising treating PBMCs from the subject ex vivo with a therapeutic agent comprising a glycan coated nanoparticle to correct a deficiency in the glycan level.

20. The method according to claim 9, comprising conjugating certain antibodies or other targeting proteins with the selected glycan ex vivo and administering the conjugated protein to the subject.

21. The method according to claim 9, comprising directly administering the selected glycan in association with an optional carrier or pharmaceutical formulation to the subject.

22. (canceled)


SEQUENCE LISTING (3 sequences)

SEQ ID NO: 1 - DNA, 22 bp, Human immunodeficiency virus type 1
gcctcaataaagcttgccttga

SEQ ID NO: 2 - DNA, 18 bp, Human immunodeficiency virus type 1
gggcgccactgctagaga

SEQ ID NO: 3 - DNA, 26 bp, Human immunodeficiency virus type 1
ccagagtcacacaacagacgggcaca